(19) United States
(12) Patent Application Publication
(10) Pub. No.: US 2009/0011426 A1
(43) Pub. Date: Jan. 8, 2009

(54) Methods of Diagnosis and Treatment of M.Tuberculosis Infection and Reagents Therefor

(76) Inventors: James A. Mackintosh, Thornleigh (AU); Robert Alan Cole, Greenwich (AU)

Correspondence Address: COZEN O'CONNOR, P.C., 1900 MARKET STREET, PHILADELPHIA, PA, 19103-3508, US

(21) Appl. No.: 11/660,851

(22) PCT Filed: Aug. 19, 2005
(86) PCT No.: PCT/AU05/01254

§ 371 Date: Dec. 21, 2007

Related U.S. Application Data
(60) Provisional application No. 60/603,243, filed on Aug. 19, 2004.

(51) Int. Cl. G01N 33/53; C07K 14/35; C07K 2/00; C07K 7/08; C07K 16/12

(52) U.S. Cl. 435/7.1; 530/350; 530/300; 530/326; 530/387.3; 530/387.9

(57) ABSTRACT

The present invention provides diagnostic, prognostic and therapeutic reagents for infection of an animal subject such as a human by M. tuberculosis, and conditions associated with such infections, such as, for example, tuberculosis. More particularly, the present invention provides a recombinant protein of M. tuberculosis designated “BSX” (SEQ ID NO: 1) and immunogenic epitopes thereof such as, for example, comprising SEQ ID NOS: 34, 25 and 45, that are useful in antibody-based diagnostic applications. The present invention also provides antibodies against BSX and its immunogenic peptides that are useful for antigen-based diagnostic and prognostic tests, and for therapy and vaccine formulations.

### FIELD OF THE INVENTION

The present invention relates to novel diagnostic, prognostic and therapeutic reagents for infection of an animal subject such as a human by M. tuberculosis, and conditions associated with such infections, such as, for example, tuberculosis. More particularly, the present invention provides the first enabling disclosure of the expression in an infected subject of a protein of M. tuberculosis designated “BSX” (SEQ ID NO: 1) and immunogenic epitopes thereof suitable for the preparation of immunological reagents, such as, for example, antigenic proteins/peptides and/or antibodies, for the diagnosis, prognosis and therapy of infection, and vaccine development.

### BACKGROUND OF THE INVENTION

### 1. General Information

As used herein the term “derived from” shall be taken to indicate that a specified integer may be obtained from a particular source albeit not necessarily directly from that source.

Unless the context requires otherwise or specifically stated to the contrary, integers, steps, or elements of the invention recited herein as singular integers, steps or elements clearly encompass both singular and plural forms of the recited integers, steps or elements.

The embodiments of the invention described herein with respect to any single embodiment and, in particular, with respect to any protein or a use thereof in the diagnosis, prognosis or therapy of M. tuberculosis shall be taken to apply mutatis mutandis to any other embodiment of the invention described herein.

The diagnostic embodiments described here for individual subjects clearly apply mutatis mutandis to the epidemiology of a population, racial group or sub-group or to the diagnosis or prognosis of individuals having a particular MHC restriction. All such variations of the invention are readily derived by the skilled artisan based upon the subject matter described herein.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific examples described herein. Functionally equivalent products, compositions and methods are clearly within the scope of the invention, as described herein.

The present invention is performed without undue experimentation using, unless otherwise indicated, conventional techniques of molecular biology, microbiology, proteomics, virology, recombining DNA technology, peptide synthesis in solution, solid phase peptide synthesis, and immunology. Such procedures are described, for example, in the following texts that are incorporated by reference:
1. Sambrook, Fritsch & Maniatis, Molecular Cloning: A Laboratory Manual, Cold Spring Harbor Laboratories, New York, Second Edition (1989), whole of Vols I, II, and III;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
4. Nucleic Acid Hybridization: A Practical Approach (B. D. Hames & S. J. Higgins, eds., 1985) IRL Press, Oxford, whole of text;
5. Immobilized Cells and Enzymes: A Practical Approach (1986) IRL Press, Oxford, whole of text;
6. Perbal, B., A Practical Guide to Molecular Cloning (1984);
7. Methods In Enzymology (S. Colowick and N. Kaplan, eds., Academic Press, Inc.), whole of series;
8. J. F. Ramalho Ortigao, “The Chemistry of Peptide Synthesis” In: Knowledge database of Access to Virtual Laboratory website (Interactiva, Germany);
9. Sakakibara, D., Teichman, J., Lien, E. Land Fenichel, R. L. (1976). Biochem. Biophys. Res. Commun. 73 336-342
10. Merrifield, R. B. (1963). J. Am. Chem. Soc. 85, 2149-2154.
11. Barany, G. and Merrifield, R. B. (1979) in The Peptides (Gross, E. and Meienhofer, J. eds.), vol. 2, pp. 1-284, Academic Press, New York.
12. Wünsch, E., ed. (1974) Synthese von Peptiden in Houben-Weyls Metoden der Organischen Chemie (Müler, E., ed.), vol. 15, 4th edn., Parts 1 and 2, Thieme, Stuttgart.
13. Bodanszky, M. (1984) Principles of Peptide Synthesis, Springer-Verlag, Heidelberg.
14. Bodanszky, M. & Bodanszky, A. (1984) The Practice of Peptide Synthesis, Springer-Verlag, Heidelberg.
15. Bodanszky, M. (1985) Int. J. Peptide Protein Res. 25, 449-474.
16. Handbook of Experimental Immunology, Vols. I-IV (D. M. Weir and C. C. Blackwell, eds., 1986, Blackwell Scientific Publications).
17. Wilkins M. R., Williams K. L., Appel R. D. and Hochstrasser (Eds) 1997 Proteome Research New Frontiers in Functional Genomics Springer, Berlin.

### 2. Description of the Related Art

Tuberculosis is a chronic, infectious disease that is generally caused by infection with Mycobacterium tuberculosis. It is a major disease in developing countries, as well as an increasing problem in developed areas of the world, with about eight million new cases and three million deaths each year. Although the infection may be asymptomatic for a considerable period of time, the disease is most commonly manifested as an acute inflammation of the lungs, resulting in fever and a productive cough. If left untreated, M. tuberculosis infection may progress beyond the primary infection site in the lungs to any organ in the body and generally results in serious complications and death.

The problems of the rapidly growing global incidence of tuberculosis and microbial resistance have been often described by many workers in the health care industry and are well known to skilled artisans in that field. In particular there is a growing recognition that new diagnostics, drugs and vaccines are urgently needed.

The immunogical mechanisms by which M. tuberculosis maintains and multiplies within the host are poorly understood. Consequently, any new information regarding the immunological relationship between tuberculosis and the host could clearly be used in many different ways to improve diagnosis, therapy and treatment of that disease.

The incidence of tuberculosis is especially common in late-staging AIDS patients, a majority of whom suffer from it. In fact, HIV infection is a most important risk factor for the development of active tuberculosis in purified protein derivative (PPD)-tuberculin-positive subjects, and the risk of acquisition of tuberculosis infection in HIV-infected immune-suppressed individuals may be markedly enhanced compared to those individuals that are not HIV-infected. It is also likely that co-infections with HIV-1 and M. tuberculosis mediate a shortened HIV symptom-free period and shortened survival time in subjects, possibly by triggering increased viral replication and virus load that results in depletion of CD4+ T-cells and immune deficiency or immune suppression (Corbett et al 2003; Ho, Mem. Inst. Oswaldo Cruz, 91, 385-387, 1996).

The sequencing of the Mycobacterium tuberculosis genome has facilitated an enormous research effort to identify potential M. tuberculosis proteins that theoretically may be expressed by the organism. However, sequence data alone are insufficient to conclude that any particular protein is expressed in vivo by the organism, let alone during infection of a human or other animal subject. Nor does the elucidation of open reading frames in the genome of M. tuberculosis indicate that any particular protein encoded or actually expressed by the bacterium comprises any immunodominant B-cell epitopes or T-cell epitopes that are required for the preparation of diagnostic, prognostic and therapeutic immunological reagents. For example, to conclude that a particular protein of M. tuberculosis or a peptide fragment derived there from has efficacy as a diagnostic reagent in an immunoassay format, or is suitable for use in a vaccine preparation, it is necessary to show that the protein is expressed during infectious cycle of the bacterium, and that the host organism mounts an immune response to the protein, and/or to a peptide fragment that comprises a B cell epitope or T-cell epitope (e.g., CD8⁺-restricted CTL epitope).

The ability to grow M. tuberculosis in culture has provided a convenient model to identify expressed tuberculosis proteins in vitro. However, the culture environment is markedly different to the environment of a human macrophage, lung, or extrapulmonary site where M. tuberculosis is found in vivo. Recent evidence indicates that the protein expression profile of intracellular parasites, such as, for example, M. tuberculosis, varies markedly depending on environmental cues, such that the expression profile of the organism in vitro may not accurately reflect the expression profile of the organism in situ.

Infection with M. tuberculosis bacilli, or reactivation of a latent infection, induces a host response comprising the recruitment of monocytes and macrophages to the site of infection. As more immune cells accumulate a nodule of granulomata forms comprising immune cells and host tissue that have been destroyed by the cytotoxic products of macrophages. As the disease progresses, macrophage enzymes cause the hydrolysis of protein, lipid and nucleic acids resulting in liquefaction of surrounding tissue and granuloma formation. Eventually the lesion ruptures and the bacilli are released into the surrounding lung, blood or lymph system.

During this infection cycle, the bacilli are exposed to four distinct host environments, being alveoli macrophage, caseous granuloma, extracellular lung and extrapulmonary sites, such as, for example the kidneys or peritoneal cavities, lymph, bone, or spine.

It is thought that bacilli can replicate to varying degrees in all these environments, however, little is known about the environmental conditions at each site. All four host environments are distinct, suggesting that the expression profile of M. tuberculosis in each environment will be different.

Accordingly, the identification of M. tuberculosis proteins from logarithmic phase cultures does not necessarily suggest which proteins are expressed or highly immunogenic in each environment in vivo. Similarly, the identification of M. tuberculosis proteins in a macrophage grown in vitro will not necessarily emulate the protein expression profile of M. tuberculosis in caseous granuloma, highly aerated lung, or at an extrapulmonary site having a low oxygen content.

Furthermore, M. tuberculosis infection within the host can be seen as a dynamic event where the host immune system is continually trying to encapsulate and destroy bacilli through destruction of infected macrophages. Consequently, the M. tuberculosis bacilli progress through cycles of intracellular growth, destruction (where both intracellular and secreted bacterial proteins are exposed and destroyed), and rapid extracellular multiplication. Host and pathogen interaction is a result of many factors, which can not be replicated in vitro.

Accordingly, until the present invention, it was not clear which M. tuberculosis proteins were the most highly expressed and/or highly immunologically active or immunogenic proteins of M. tuberculosis in any particular environment in vivo.

There clearly remains a need for rapid and cost-effective diagnostic and prognostic reagents for determining infection by M. tuberculosis and/or disease conditions associated therewith.

### SUMMARY OF INVENTION

In work leading up to the present invention, the inventors sought to elucidate the range of proteins expressed by M. tuberculosis in a range of in vivo environments, to thereby identify highly expressed and/or highly immunogenic M. tuberculosis proteins.

The inventors used a proteomics approach to identify M. tuberculosis proteins in the body fluids of a cohort of diseased patients, including sputum, pleural fluid, plasma and serum. A highly-expressed M. tuberculosis protein was identified in vivo in samples of a cohort of M. tuberculosis-infected patients, having high amino acid sequence identity to a sequence postulated to be encoded by the M. tuberculosis genome. The protein identified by the inventors comprised the amino acid sequence set forth in SEQ ID NO: 1. In mass screens, 47% of the TB-positive subjects were shown to produce antibody responses against the protein, compared to only 6% of the TB-negative subjects, indicating that the presence of the protein is correlated with a TB diagnosis.

The amino acid sequence set forth in SEQ ID NO: 1 was designated “BSX”. Sequence analysis indicated that the sequence of the M. tuberculosis protein BSX comprised a helix-turn-helix motif found in XRE-family like proteins (i.e., transcriptional regulatory proteins that bind to cis-acting xenobiotic response elements in the upstream regions of certain prokaryote genes (e.g., Cro, cI, HipB).

The inventors further produced a PEPSET comprising 43 synthetic overlapping peptides (i.e., SEQ ID Nos: 2-44) covering the amino acid sequence of SEQ ID NO: 1, to determine whether the identified protein was present across geographic and racial boundaries, and to identify B-cell epitopes for subsequent monoclonal and polyclonal antibody production. TB-negative sera, and sera from TB-positive subjects, including TB patients who are seronegative or seropositive for human immunodeficiency virus (HIV-1 and/or HIV-2) i.e., HIV⁺/TB⁺ and HIV⁻/TB⁺ subjects, were screened for the presence of antibodies to each peptide in the PEPSET. Six of these peptides were immunogenic in the TB-positive cohort. A high proportion (80%) of HIV⁺/TB⁺ subjects, compared to only 25% of HIV⁻/TB⁺ subjects, possessed antibodies to eight (8) peptides. All of these peptides (SEQ ID NOs: 14, 20, 21, 22, 24, 25, and 36) did not appear in the controls, indicating that they are useful as diagnostic indicators of a latent or active infection by M. tuberculosis, especially in an HIV-infected individual.

Antibodies against the amino acid sequence set forth in SEQ ID NO: 1 or a B-cell epitope thereof were further shown by the inventors to be present in subjects during extrapulmonary infection by M. tuberculosis, in at least one population. The detection of such antibodies was therefore examined by the inventors as a suitable assay readout for the diagnosis of tuberculosis. In this respect, the inventors determined that recombinant BSX protein comprising the sequence set forth in SEQ ID NO: 1 and peptides comprising the immunodominant B-cell epitope within SEQ ID NO: 24-25 (e.g., a peptide comprising the sequence set forth in SEQ ID NO: 45) are useful in antibody-based diagnostic tests for tuberculosis, including multianalyte tests, by virtue of their high sensitivity and specificity. Other peptides derived from the full-length sequence of the BSX protein, e.g., peptides comprising the sequences set forth in SEQ ID NOs: 46 and 47, are useful for such tests, e.g., as secondary ligands in a multi-analyte assay format, by virtue of their high specificity.

The inventors have also made antibodies against BSX-derived peptides for the development of antigen-based diagnostic and prognostic assays. Plasma cytomas were produced that express antibodies against selected peptides. These cytomas can be used, for example, to produce hybridomas expressing monoclonal antibodies that bind to a B-cell epitope of M. tuberculosis BSX protein in patient samples, thereby detecting the bacterium. Additional antibodies are also obtained with a view to selecting high-affinity antibodies capable of detecting M. tuberculosis BSX protein in serum at sub-nanogram/ml or sub-picogram/ml levels in patient sera.

These findings have provided the means for producing novel diagnostics for the detection of M. tuberculosis infection in a subject, and novel prognostic indicators for the progression of infection or a disease state associated therewith. Preferably, the BSX protein or a B-cell epitope thereof is useful for the early diagnosis of infection or disease. It will also be apparent to the skilled person that such prognostic indicators as described herein may be used in conjunction with therapeutic treatments for tuberculosis or an infection associated therewith.

Accordingly, the present invention provides the means for producing novel diagnostics for the detection of M. tuberculosis infection in a subject, and novel prognostic indicators for the progression of infection or a disease state associated therewith, either by detecting BSX solus or as part of a multi-analyte test. Preferably, the BSX protein or a B-cell epitope thereof is useful for the early diagnosis of infection or disease. It will also be apparent to the skilled person that such prognostic indicators as described herein may be used in conjunction with therapeutic treatments for tuberculosis or an infection associated therewith.

For example, the present invention provides an isolated or recombinant immunogenic BSX protein of Mycobacterium tuberculosis or an immunogenic BSX peptide or immunogenic BSX fragment or epitope thereof.

Preferably, the isolated or recombinant immunogenic BSX protein of M. tuberculosis comprises the amino acid sequence set forth in SEQ ID NO: 1 or having an amino acid sequence that is at least about 95% identical to SEQ ID NO: 1.

Preferably, the immunogenic BSX peptide is a synthetic peptide. Preferably the BSX peptide, fragment or epitope comprises at least about 5 consecutive amino acid residues of the sequence set forth in SEQ ID NO: 1, more preferably at least about 10 consecutive amino acid residues of the sequence set forth in SEQ ID NO: 1, even more preferably at least about 15 consecutive amino acid residues of the sequence set forth in SEQ ID NO: 1, and still more preferably at least about 5 consecutive amino acid residues of the sequence set forth in SEQ ID NO: 1 fused to about 1-5 additional amino acid residues at the N-terminus and/or the C-terminus.

In a particularly preferred embodiment, the BSX peptide, fragment or epitope comprises an amino acid sequence set forth in any one of SEQ ID Nos: 2-53 and preferably, a sequence selected from the group consisting of SEQ ID NOs: 14, 20, 21, 22, 24, 25, 36, 45, 46 and 47, and more preferably a sequence selected from the group consisting of SEQ ID NOs: 24, 25, 45, 46 and 47, and still more preferably a sequence selected from the group consisting of SEQ ID NO: 25 and SEQ ID NO: 45, or an immunologically cross-reactive variant of any one of said sequences that comprises an amino acid sequence that is at least about 95% identical thereto.

It will be apparent from the disclosure that a preferred immunogenic BSX peptide, fragment or epitope comprises an amino acid sequence of at least about 5 consecutive amino acid residues positioned between about residue 65 to about residue 84 of SEQ ID NO: 1, more preferably at least about 5 consecutive amino acid residues positioned between about residue 65 to about residue 75 of SEQ ID NO: 1. Still more preferably, a preferred immunogenic BSX peptide, fragment or epitope comprises an amino acid sequence of at least about 5 consecutive amino acid residues positioned between residue 67 to residue 73 of SEQ ID NO: 1, corresponding to at least 5 consecutive residues of the sequence set forth in SEQ ID NO: 45. This includes any peptides comprising an N-terminal extension of up to about 5 amino acid residues in length and/or a C-terminal extension of up to about 5 amino acid residues in length.

It is clearly within the scope of the present invention for the isolated or recombinant immunogenic BSX protein of Mycobacterium tuberculosis or an immunogenic BSX peptide or immunogenic BSX fragment or epitope thereof to comprise one or more labels or detectable moieties e.g., to facilitate detection or isolation or immobilization. Preferred labels include, for example, biotin, glutathione-S-transferase (GST), FLAG epitope, hexa-histidine, β-galactosidase, horseradish peroxidase, streptavidin or gold.

The present invention also provides a fusion protein comprising one or more immunogenic BSX peptides, fragments or epitopes according to any embodiment described herein. For example, the N-terminal and C-terminal portions of BSX protein can be fused such as provided in SEQ ID NO: 46 wherein seven N-terminal residues and seven C-terminal residues are fused via an internal cysteine residue. The skilled artisan will be aware that such an internal linking residue is optional or preferred and not essential to the production, or every use, of a fusion protein. However, preferred fusion proteins may comprise a linker separating an immunogenic BSX peptide from one or more other peptide moieties, such as, for example, a single amino acid residue (e.g., glycine, cysteine, lysine), a peptide linker (e.g., a non-immunogenic peptide such as a poly-lysine or poly-glycine), poly-carbon linker comprising up to about 6 or 8 or 10 or 12 carbon residues, or a chemical linker. Such linkers may facilitate antibody production or vaccine formulation e.g., by permitting linkage to a lipid or hapten, or to permit cross-linking or binding to a ligand. The expression of proteins as fusions may also enhance their solubility.

Preferred fusion proteins will comprise the BSX protein, peptide, fragment or epitope fused to a carrier protein, detectable label or reporter molecule e.g., glutathione-S-transferase (GST), FLAG epitope, hexa-histidine, β-galactosidase, thioredoxin (TRX) (La Vallie et al., Bio/Technology 11, 187-193, 1993), maltose binding protein (MBP), Escherichia coli NusA protein (Fayard, E. M. S., Thesis, University of Oklahoma, USA, 1999; Harrison, inNovations 11, 4-7, 2000), E. coli BFR (Harrison, inNovations 11, 4-7, 2000) and E. coli GrpE (Harrison, inNovations 11, 4-7, 2000).

The present invention also provides an isolated protein aggregate comprising one or more immunogenic BSX peptides, fragments or epitopes according to any embodiment described herein. Preferred protein aggregates will comprise the protein, peptide, fragment or epitope complexed to an immunoglobulin e.g., IgA, IgM or IgG, such as, for example as a circulating immune complex (CIC). Exemplary protein aggregates may be derived, for example, derived from an antibody-containing biological sample of a subject.

The present invention also encompasses the use of the isolated or recombinant immunogenic BSX protein of Mycobacterium tuberculosis or an immunogenic BSX peptide or immunogenic BSX fragment or epitope thereof according to any embodiment described herein for detecting a past or present infection or latent infection by M. tuberculosis in a subject, wherein said infection is determined by the binding of antibodies in a sample obtained from the subject to said isolated or recombinant immunogenic BSX protein or an immunogenic BSX peptide or immunogenic BSX fragment or epitope.

The present invention also encompasses the use of the isolated or recombinant immunogenic BSX protein of Mycobacterium tuberculosis or an immunogenic BSX peptide or immunogenic BSX fragment or epitope thereof according to any embodiment described herein for eliciting the production of antibodies that bind to M. tuberculosis BSX.

The present invention also encompasses the use of the isolated or recombinant immunogenic BSX protein of Mycobacterium tuberculosis or an immunogenic BSX peptide or immunogenic BSX fragment or epitope thereof according to any embodiment described herein in the preparation of a medicament for immunizing a subject against infection by M. tuberculosis.

The present invention also provides a pharmaceutical composition comprising the isolated or recombinant immunogenic BSX protein of Mycobacterium tuberculosis or an immunogenic BSX peptide or immunogenic BSX fragment or epitope thereof according to any embodiment described herein in combination with a pharmaceutically acceptable diluent, e.g., an adjuvant.

The present invention also provides an isolated nucleic acid encoding the isolated or recombinant immunogenic BSX protein of Mycobacterium tuberculosis or an immunogenic BSX peptide or immunogenic BSX fragment or epitope thereof according to any embodiment described herein e.g., for the preparation of nucleic acid based vaccines or for otherwise expressing the immunogenic polypeptide, protein, peptide, fragment or epitope.

The present invention also provides a cell expressing the isolated or recombinant immunogenic BSX protein of Mycobacterium tuberculosis or an immunogenic BSX peptide or immunogenic BSX fragment or epitope thereof according to any embodiment described herein. The cell may preferably consist of an antigen-presenting cell (APC) that expresses the isolated or recombinant immunogenic BSX protein of Mycobacterium tuberculosis or an immunogenic BSX peptide or immunogenic BSX fragment or epitope thereof e.g., on its surface.

The present invention also provides an isolated or recombinant antibody or immune reactive fragment of an antibody that binds specifically to the isolated or recombinant immunogenic BSX protein of Mycobacterium tuberculosis or an immunogenic BSX peptide or immunogenic BSX fragment or epitope thereof according to any embodiment described herein, or to a fusion protein or protein aggregate comprising said immunogenic BSX protein, peptide, fragment or epitope. Preferred antibodies include, for example, a monoclonal or polyclonal antibody preparation. This extends to any isolated antibody-producing cell or antibody-producing cell population, e.g., a hybridoma or plasmacytoma producing antibodies that bind to a BSX protein or immunogenic fragment of a BSX protein or other immunogenic peptide comprising a sequence derived from the sequence of a BSX protein.

The present invention also provides for the use of the isolated or recombinant antibody according to any embodiment described herein or an immune-reactive fragment thereof in medicine.

The present invention also provides for the use of the isolated or recombinant antibody according to any embodiment described herein or an immune-reactive fragment thereof for detecting a past or present (i.e., active) infection or a latent infection by M. tuberculosis in a subject, wherein said infection is determined by the binding of the antibody or fragment to M. tuberculosis BSX protein or an immunogenic fragment or epitope thereof present in a biological sample obtained from the subject.

The present invention also provides for the use of the isolated or recombinant antibody according to any embodiment described herein or an immune-reactive fragment thereof for identifying the bacterium M. tuberculosis or cells infected by M. tuberculosis or for sorting or counting of said bacterium or said cells.

The isolated or recombinant antibodies, or immune-reactive fragments thereof, are also useful in therapeutic, diagnostic and research applications for detecting a past or present infection, or a latent infection, by M. tuberculosis as determined by the binding of the antibody to an M. tuberculosis BSX protein or an immunogenic fragment or epitope thereof present in a biological sample from a subject (i.e., an antigen-based immunoassay).

Other applications of the subject antibodies include the purification and study of the diagnostic/prognostic BSX protein, identification of cells infected with M. tuberculosis, or for sorting or counting of such cells.

The antibodies and fragments thereof are also useful in therapy, including prophylaxis, diagnosis, or prognosis, and the use of such antibodies or fragments for the manufacture of a medicament for use in treatment of infection by M. tuberculosis. For example, specific humanized antibodies or ligands are produced that bind and neutralize a BSX protein or M. tuberculosis, especially in vivo. The humanized antibodies or ligands are used as in the preparation of a medicament for treating TB-specific disease or M. tuberculosis infection in a human subject, such as, for example, in the treatment of an active or chronic M. tuberculosis infection.

The present invention also provides a composition comprising the isolated or recombinant antibody according to any embodiment described herein and a pharmaceutically acceptable carrier, diluent or excipient.

The present invention also provides a method of diagnosing tuberculosis or an infection by M. tuberculosis in a subject comprising detecting in a biological sample from said subject antibodies against an immunogenic BSX protein or an immunogenic BSX peptide or immunogenic BSX fragment or epitope thereof, the presence of said antibodies in the sample is indicative of infection. In a related embodiment, the presence of said antibodies in the sample is indicative of infection. The infection may be a past or active infection, or a latent infection, however this assay format is particularly useful for detecting active infection and/or recent infection.

For example, the method may be an immunoassay, e.g., comprising contacting a biological sample derived from the subject with the isolated or recombinant immunogenic BSX protein of Mycobacterium tuberculosis or an immunogenic BSX peptide or immunogenic BSX fragment or epitope thereof according to any embodiment described herein (e.g., a peptide comprising an amino acid sequence set forth in any one of SEQ ID Nos: 2-53 and preferably, a sequence selected from the group consisting of SEQ ID NOs: 14, 20, 21, 22, 24, 25, 36, 45, 46 and 47, and more preferably a sequence selected from the group consisting of SEQ ID NOs: 24, 25, 45, 46 and 47, and still more preferably a sequence selected from the group consisting of SEQ ID NO: 25 and SEQ ID NO: 45, or an immunologically cross-reactive variant of any one of said sequences that comprises an amino acid sequence that is at least about 95% identical thereto) for a time and under conditions sufficient for an antigen-antibody complex to form and then detecting the formation of an antigen-antibody complex. The sample is an antibody-containing sample e.g., a sample that comprises blood or serum or an immunoglobulin fraction obtained from the subject. The sample may contain circulating antibodies in the form of complexes with BSX antigenic fragments. Generally, the antigen-antibody complex will be detected in such assay formats using antibodies capable of binding to the patient's immunoglobulin e.g., anti-human Ig antibodies.

It is within the scope of the present invention to include a multi-analyte test in this assay format, wherein multiple antigenic epitopes are used to confirm a diagnosis obtained using a BSX peptide. For example, the patient sample may be contacted with BSX or immunogenic BSX peptide or fragment or epitope and with a M. tuberculosis glutamine synthase (GS) protein (e.g., SwissProt Database Accession No. 033342) or immunogenic peptide derived there from, e.g., a peptide derived from a surface-exposed region of a GS protein, or comprising the sequence RGTDGSAVFADSNGPHGMSSMFRSFC (SEQ ID NO: 54) or WASGYRGLTPASDYNIDYAIC (SEQ ID NO: 55). Immunogenic M. tuberculosis GS and peptide derivatives for detecting tuberculosis or infection by M. tuberculosis are also described in detail in the instant applicant's co-pending International Patent Application No. PCT/AU2005/000930 filed Jun. 24, 2005 the disclosure of which is incorporated herein in its entirety. Assays for a secondary analyte e.g., antibodies against glutamine synthetase are conveniently performed in the same manner as for detecting antibodies against BSX in serum. The assays may be performed simultaneously or at different times, and using the same or different patient samples. The assays may also be performed in the same reaction vessel, provided that different detection systems are used to detect the different antibodies, e.g., anti-human Ig labelled using different reporter molecules such as different coloured dyes, fluorophores, radionucleotides or enzymes.

As used herein, the term “infection” shall be understood to mean invasion and/or colonisation by a microorganism and/or multiplication of a micro-organism, in particular, a bacterium or a virus, in the respiratory tract of a subject. Such an infection may be unapparent or result in local cellular injury. The infection may be localised, subclinical and temporary or alternatively may spread by extension to become an acute or chronic clinical infection. The infection may also be a past infection wherein residual BSX antigen, or alternatively, reactive host antibodies that bind to isolated BSX protein or peptides, remain in the host. The infection may also be a latent infection, in which the microorganism is present in a subject, however the subject does not exhibit symptoms of disease associated with the organism. Preferably, the infection is a pulmonary or extra-pulmonary infection by M. tuberculosis, and more preferably an extra-pulmonary infection. By “pulmonary” infection is meant an infection of the airway of the lung, such as, for example, an infection of the lung tissue, bronchi, bronchioles, respiratory bronchioles, alveolar ducts, alveolar sacs, or alveoli. By “extra-pulmonary” is meant outside the lung, encompassing, for example, kidneys, lymph, urinary tract, bone, skin, spinal fluid, intestine, peritoneal, pleural and pericardial cavities.

The present invention also provides a method of diagnosing tuberculosis or infection by M. tuberculosis in a subject comprising detecting in a biological sample from said subject an immunogenic BSX protein or an immunogenic BSX peptide or immunogenic BSX fragment or epitope thereof, wherein the presence of said protein or immunogenic fragment or epitope in the sample is indicative of disease, disease progression or infection. In a related embodiment, the presence of said protein or immunogenic fragment or epitope in the sample is indicative of infection. For example, the method can comprise an immunoassay e.g., contacting a biological sample derived from the subject with one or more antibodies capable of binding to a BSX protein or an immunogenic fragment or epitope thereof, and detecting the formation of an antigen-antibody complex. In a particularly preferred embodiment, an antibody is an isolated or recombinant antibody or immune reactive fragment of an antibody that binds specifically to the isolated or recombinant immunogenic BSX protein of Mycobacterium tuberculosis or an immunogenic BSX peptide or immunogenic BSX fragment or epitope thereof according to any embodiment described herein or to a fusion protein or protein aggregate comprising said immunogenic BSX protein, peptide, fragment or epitope. The diagnostic assay of the present invention is particularly useful for detecting TB in a subject that is immune compromised or immune deficient, e.g., a subject that is HIV+. The samples used for conducting such assays include, for example, (i) an extract from a tissue selected from the group consisting of brain, breast, ovary, lung, colon, pancreas, testes, liver, muscle, bone and mixtures thereof; (ii) body fluid(s) selected from the group consisting of sputum, serum, plasma, whole blood, saliva, urine, pleural fluid and mixtures thereof, and (iii) samples derived from body fluid(s) selected from the group consisting of sputum, serum, plasma, whole blood, saliva, urine, pleural fluid and mixtures thereof.

The present invention also provides a method for determining the response of a subject having tuberculosis or an infection by M. tuberculosis to treatment with a therapeutic compound for said tuberculosis or infection, said method comprising detecting a BSX protein or an immunogenic fragment or epitope thereof in a biological sample from said subject, wherein a level of the protein or fragment or epitope that is enhanced compared to the level of that protein or fragment or epitope detectable in a normal or healthy subject indicates that the subject is not responding to said treatment or has not been rendered free of disease or infection. For example, the method can comprise an immunoassay e.g., contacting a biological sample derived from the subject with one or more antibodies capable of binding to a BSX protein or an immunogenic fragment or epitope thereof, and detecting the formation of an antigen-antibody complex. In a particularly preferred embodiment, an antibody is an isolated or recombinant antibody or immune reactive fragment of an antibody that binds specifically to the isolated or recombinant immunogenic BSX protein of Mycobacterium tuberculosis or an immunogenic BSX peptide or immunogenic BSX fragment or epitope thereof according to any embodiment described herein or to a fusion protein or protein aggregate comprising said immunogenic BSX protein, peptide, fragment or epitope. The diagnostic assay of the present invention is particularly useful for detecting TB in a subject that is immune compromised or immune deficient, e.g., a subject that is HIV+. The samples used for conducting such assays include, for example, (i) an extract from a tissue selected from the group consisting of brain, breast, ovary, lung, colon, pancreas, testes, liver, muscle, bone and mixtures thereof; (ii) body fluid(s) selected from the group consisting of sputum, serum, plasma, whole blood, saliva, urine, pleural fluid and mixtures thereof; and (iii) samples derived from body fluid(s) selected from the group consisting of sputum, serum, plasma, whole blood, saliva, urine, pleural fluid and mixtures thereof.

The present invention also provides a method for determining the response of a subject having tuberculosis or an infection by M. tuberculosis to treatment with a therapeutic compound for said tuberculosis or infection, said method comprising detecting a BSX protein or an immunogenic fragment or epitope thereof in a biological sample from said subject, wherein a level of the protein or fragment or epitope that is lower than the level of the protein or fragment or epitope detectable in a subject suffering from tuberculosis or infection by M. tuberculosis indicates that the subject is responding to said treatment or has been rendered free of disease or infection. For example, the method can comprise an immunoassay e.g., contacting a biological sample derived from the subject with one or more antibodies capable of binding to a BSX protein or an immunogenic fragment or epitope thereof, and detecting the formation of an antigen-antibody complex. In a particularly preferred embodiment, an antibody is an isolated or recombinant antibody or immune reactive fragment of an antibody that binds specifically to the isolated or recombinant immunogenic BSX protein of Mycobacterium tuberculosis or an immunogenic BSX peptide or immunogenic BSX fragment or epitope thereof according to any embodiment described herein or to a fusion protein or protein aggregate comprising said immunogenic BSX protein, peptide, fragment or epitope. The diagnostic assay of the present invention is particularly useful for detecting TB in a subject that is immune compromised or immune deficient, e.g., a subject that is HIV+. The samples used for conducting such assays include, for example, (i) an extract from a tissue selected from the group consisting of brain, breast, ovary, lung, colon, pancreas, testes, liver, muscle, bone and mixtures thereof; (ii) body fluid(s) selected from the group consisting of sputum, serum, plasma, whole blood, saliva, urine, pleural fluid and mixtures thereof; and (iii) samples derived from body fluid(s) selected from the group consisting of sputum, serum, plasma, whole blood, saliva, urine, pleural fluid and mixtures thereof.

The present invention also provides a method of monitoring disease progression, responsiveness to therapy or infection status by M. tuberculosis in a subject comprising determining the level of a BSX protein or an immunogenic fragment or epitope thereof in a biological sample from said subject at different times, wherein a change in the level of the BSX protein, fragment or epitope indicates a change in disease progression, responsiveness to therapy or infection status of the subject. In a preferred embodiment, the method further comprises administering a compound for the treatment of tuberculosis or infection by M. tuberculosis when the level of BSX protein, fragment or epitope increases over time. For example, the method can comprise an immunoassay e.g., contacting a biological sample derived from the subject with one or more antibodies capable of binding to a BSX protein or an immunogenic fragment or epitope thereof, and detecting the formation of an antigen-antibody complex. In a particularly preferred embodiment, an antibody is an isolated or recombinant antibody or immune reactive fragment of an antibody that binds specifically to the isolated or recombinant immunogenic BSX protein of Mycobacterium tuberculosis or an immunogenic BSX peptide or immunogenic BSX fragment or epitope thereof according to any embodiment described herein or to a fusion protein or protein aggregate comprising said immunogenic BSX protein, peptide, fragment or epitope. The diagnostic assay of the present invention is particularly useful for detecting TB in a subject that is immune compromised or immune deficient, e.g., a subject that is HIV+. The samples used for conducting such assays include, for example, (i) an extract from a tissue selected from the group consisting of brain, breast, ovary, lung, colon, pancreas, testes, liver, muscle, bone and mixtures thereof; (ii) body fluid(s) selected from the group consisting of sputum, serum, plasma, whole blood, saliva, urine, pleural fluid and mixtures thereof; and (iii) samples derived from body fluid(s) selected from the group consisting of sputum, serum, plasma, whole blood, saliva, urine, pleural fluid and mixtures thereof.

In a particularly preferred embodiment, circulating immune complexes (CICs) are detected in an antigen-based assay platform or antibody-based assay platform. For antigen-based assay platforms, the detection of CICs may provide a significant signal amplification over the detection of isolated antigen in circulation, by virtue of detecting the immunoglobulin moiety of the CIC. In accordance with this embodiment, a capture reagent e.g., a capture antibody is used to capture the BSX antigen (BSX polypeptide or an immune reactive fragment or epitope thereof) complexed with the subject's immunoglobulin, in addition to isolated antigen in the subject's circulation. Anti-Ig antibodies, optionally conjugated to a detectable label, are used to specifically bind the captured CIC thereby detecting CIC patient samples. Within the scope of this invention, the anti-Ig antibody binds preferentially to IgM, IgA or IgG in the sample. In a particularly preferred embodiment, the anti-Ig antibody binds to human Ig, e.g., human IgA, human IgG or human IgM. The anti-Ig antibody may be conjugated to any standard detectable label known in the art. This is particularly useful for detecting infection by a pathogenic agent, e.g., a bacterium or virus, or for the diagnosis of any disease or disorder associated with CICs. Accordingly, the diagnostic methods described according to any embodiment herein are amenable to a modification wherein the sample derived from the subject comprises one or more circulating immune complexes comprising immunoglobulin (Ig) bound to BSX protein of Mycobacterium tuberculosis or one or more immunogenic BSX peptides, fragments or epitopes thereof and wherein detecting the formation of an antigen-antibody complex comprises contacting an anti-Ig antibody with an immunoglobulin moiety of the circulating immune complex(es) for a time and under conditions sufficient for a complex to form than then detecting the bound anti-Ig antibody.

It is also within the scope of the present invention to include a multi-analyte test in one or more of the preceding antigen-based assay formats, wherein multiple antibodies of different specificities are used to confirm a diagnosis obtained using anti-BSX antibodies, thereby enhancing specificity and/or selectivity. For example, the patient sample may be contacted with antibodies against BSX or immunogenic BSX peptide or fragment or epitope and antibodies against M. tuberculosis glutamine synthase (GS) protein (e.g., SwissProt Database Accession No. 033342) or immunogenic peptide derived there from, e.g., antibodies prepared against a peptide derived from a surface-exposed region of a GS protein or comprising the sequence RGTDGSAVFADSNGPHGMSSMFRSFC (SEQ ID NO: 54) or WASGYRGLTPASDYNIDYAIC (SEQ ID NO: 55). Antibodies against immunogenic M. tuberculosis GS peptides are also described in detail in the instant applicant's co-pending International Patent Application No. PCT/AU2005/000930 filed Jun. 24, 2005 the disclosure of which is incorporated herein in its entirety. The antigen-antibody complexes formed are then detected using antibodies capable of binding to each protein analyte (e.g., anti-BSX and anti-GS antibodies), or in the case of CIC detections, antibodies capable of binding to human immunoglobulins. The assays may be performed simultaneously or at different times, and using the same or different patient samples. The assays may also be performed in the same reaction vessel, provided that different detection systems are used to detect the different antigens or CICs comprising the different antigens, e.g., anti-human Ig labelled using different reporter molecules such as different coloured dyes, fluorophores, radionucleotides or enzymes; or differentially-labelled anti-BSX and anti-GS antibodies. As with other immunoassays described herein, the secondary antibody is optionally conjugated to a suitable detectable label e.g., horseradish peroxidase (HRP) or β-galactosidase or β-glucosidase, colloidal gold particles, amongst others. Standard methods for employing such labels in the detection of the complexes formed will be apparent to the skilled artisan.

The present invention also provides a method of treatment of tuberculosis or infection by M. tuberculosis comprising:
(i) performing a diagnostic method according to any embodiment described herein thereby detecting the presence of M. tuberculosis infection in a biological sample from a subject; and
(ii) administering a therapeutically effective amount of a pharmaceutical composition to reduce the number of pathogenic bacilli in the lung, blood or lymph system of the subject.

The present invention also provides a method of treatment of tuberculosis or infection by M. tuberculosis comprising:
(i) performing a diagnostic method according to any embodiment described herein thereby detecting the presence of M. tuberculosis infection in a biological sample from a subject being treated with a first pharmaceutical composition; and
(ii) administering a therapeutically effective amount of a second pharmaceutical composition to reduce the number of pathogenic bacilli in the lung, blood or lymph system of the subject.

The present invention also provides a method of treatment of tuberculosis in a subject comprising performing a diagnostic method or prognostic method as described herein. In one embodiment, the present invention provides a method of prophylaxis comprising:
(i) detecting the presence of M. tuberculosis infection in a biological sample from a subject; and
(ii) administering a therapeutically effective amount of a pharmaceutical composition to reduce the number of pathogenic bacilli in the lung, blood or lymph system of the subject.

More particularly, an immunogenic BSX protein or one or more immunogenic BSX peptides, fragments or epitopes thereof induce(s) the specific production of a high titer antibody when administered to an animal subject.

Accordingly, the invention also provides a method of eliciting the production of antibody against M. tuberculosis comprising administering an immunogenic BSX protein or one or more immunogenic BSX peptides or immunogenic BSX fragments or epitopes thereof to said subject for a time and under conditions sufficient to elicit the production of antibodies, such as, for example, neutralizing antibodies against M. tuberculosis.

The present invention clearly contemplates the use of an immunogenic BSX protein or one or more immunogenic BSX peptides or immunogenic BSX fragments or epitopes thereof in the preparation of a therapeutic or prophylactic subunit vaccine against M. tuberculosis infection in a human or other animal subject.

Accordingly, this invention also provides a vaccine comprising an immunogenic BSX protein or one or more immunogenic BSX peptides or immunogenic BSX fragments or epitopes thereof in combination with a pharmaceutically acceptable diluent. Preferably, the protein or peptide(s) or fragment(s) or epitope(s) thereof is(are) formulated with a suitable adjuvant.

Alternatively, the peptide or derivative or variant is formulated as a cellular vaccine via the administration of an autologous or allogeneic antigen presenting cell (APC) or a dendritic cell that has been treated in vitro so as to present the peptide on its surface.

Nucleic acid-based vaccines that comprise nucleic acid, such as, for example, DNA or RNA, encoding an immunogenic BSX protein or one or more immunogenic BSX peptides or immunogenic BSX fragments or epitopes thereof cloned into a suitable vector (e.g. vaccinia, canary pox, adenovirus, or other eukaryotic virus vector) are also contemplated. Preferably, DNA encoding an immunogenic BSX protein or an immunogenic BSX peptide or immunogenic BSX fragment or epitope thereof is formulated into a DNA vaccine, such as, for example, in combination with the existing Calmette-Guerin (BCG) or an immune adjuvant such as vaccinia virus, Freund's adjuvant or another immune stimulant.

The present invention further provides for the use of an immunogenic BSX protein or one or more immunogenic BSX peptides or one or more immunogenic BSX fragments or one or more epitopes thereof in the preparation of a composition for the prophylactic or therapeutic treatment or diagnosis of tuberculosis or infection by M. tuberculosis in a subject, such as, for example, a subject infected with HIV-1 and/or HIV-2, including the therapeutic treatment of a latent M. tuberculosis infection in a human subject.

In an alternative embodiment, the present invention provides for the use of an immunogenic BSX protein or one or more immunogenic BSX peptides or one or more immunogenic BSX fragments or one or more epitopes thereof in the preparation of a composition for the prophylactic or therapeutic treatment or diagnosis of tuberculosis or infection by M. tuberculosis in a subject wherein the subject has been subjected previously to antiviral therapy against HIV-1 and/or HIV-2.

The present invention also provides a kit for detecting M. tuberculosis infection in a biological sample, said kit comprising:
(i) one or more isolated antibodies or immune reactive fragments thereof that bind specifically to the isolated or recombinant immunogenic BSX protein of Mycobacterium tuberculosis or an immunogenic BSX peptide or immunogenic BSX fragment or epitope thereof according to any embodiment described herein or to a fusion protein or protein aggregate comprising said immunogenic BSX protein, peptide, fragment or epitope; and
(ii) means for detecting the formation of an antigen-antibody complex, optionally packaged with instructions for use.

The present invention also provides a kit for detecting M. tuberculosis infection in a biological sample, said kit comprising:
(i) isolated or recombinant immunogenic BSX protein of Mycobacterium tuberculosis or an immunogenic BSX peptide or immunogenic BSX fragment or epitope thereof according to any one of claims 1 to 16; and
(ii) means for detecting the formation of an antigen-antibody complex,
   optionally packaged with instructions for use.

The assays described herein are amenable to any assay format, and particularly to solid phase ELISA, flow through immunoassay formats, capillary formats, and for the purification or isolation of immunogenic proteins, peptides, fragments and epitopes and CICs.

Accordingly, the present invention also provides a solid matrix having adsorbed thereto an isolated or recombinant BSX protein or an immunogenic BSX peptide or immunogenic BSX fragment or epitope thereof according to any one embodiment described herein or a fusion protein or protein aggregate comprising said immunogenic BSX protein, peptide, fragment or epitope. For example, the solid matrix may comprise a membrane, e.g., nylon or nitrocellulose. Alternatively, the solid matrix may comprise a polystyrene or polycarbonate microwell plate or part thereof (e.g., one or more wells of a microtiter plate), a dipstick, a glass support, or a chromatography resin.

In an alternative embodiment, the invention also provides a solid matrix having adsorbed thereto an antibody that binds to an isolated or recombinant BSX protein or an immunogenic BSX peptide or immunogenic BSX fragment or epitope thereof according to any embodiment described herein or to a fusion protein or protein aggregate comprising said immunogenic BSX protein, peptide, fragment or epitope. For example, the solid matrix may comprise a membrane, e.g., nylon or nitrocellulose. Alternatively, the solid matrix may comprise a polystyrene or polycarbonate microwell plate or part thereof (e.g., one or more wells of a microtiter plate), a dipstick, a glass support, or a chromatography resin.

It is clearly within the scope of the present invention for such solid matrices to comprise additional antigens and/or antibodies as required to perform an assay described herein, especially for multianalyte tests employing multiple antigens or multiple antibodies.

### DETAILED DESCRIPTION OF THE PREFERRED EMBODIMENTS

### Isolated or Recombinant BSX Protein and Immunogenic Fragments and Epitopes Thereof

One aspect of the present invention provides an isolated or recombinant BSX protein or an immunogenic fragment or epitope thereof.

This aspect of the invention encompasses any synthetic or recombinant peptides derived from a BSX protein referred to herein, including the full-length BSX protein, and/or a derivative or analogue of a BSX protein or an immunogenic fragment or epitope thereof.

As used herein, the term “BSX” shall be taken to mean any peptide, polypeptide, or protein having at least about 80% amino acid sequence identity to the amino acid sequence set forth in SEQ ID NO: 1. Preferably, the percentage identity of a BSX protein to SEQ ID NO: 1 is at least about 85%, more preferably at least about 90%, even more preferably at least about 95% and still more preferably at least about 99%. The present invention is not to be restricted to the use of the exemplified M. tuberculosis BSX protein because, as will be known to those skilled in the art, it is possible to define a fragment of a protein having sequence identity and immunological equivalence to a region of the exemplified M. tuberculosis BSX protein without undue experimentation.

In determining whether or not two amino acid sequences fall within the defined percentage identity limits supra, those skilled in the art will be aware that it is possible to conduct a side-by-side comparison of the amino acid sequences. In such comparisons or alignments, differences will arise in the positioning of non-identical residues depending upon the algorithm used to perform the alignment. In the present context, references to percentage identities and similarities between two or more amino acid sequences shall be taken to refer to the number of identical and similar residues respectively, between said sequences as determined using any standard algorithm known to those skilled in the art. In particular, amino acid identities and similarities are calculated using software of the Computer Genetics Group, Inc., University Research Park, Maddison, Wisconsin, United States of America, e.g., using the GAP program of Devereaux et al., Nucl. Acids Res. 12, 387-395, 1984, which utilizes the algorithm of Needleman and Wunsch, J. Mol. Biol. 48, 443-453, 1970. Alternatively, the CLUSTAL W algorithm of Thompson et al., Nucl. Acids Res. 22, 4673-4680, 1994, is used to obtain an alignment of multiple sequences, wherein it is necessary or desirable to maximise the number of identical/similar residues and to minimise the number and/or length of sequence gaps in the alignment. Amino acid sequence alignments can also be performed using a variety of other commercially available sequence analysis programs, such as, for example, the BLAST program available at NCBI.

Particularly preferred fragments include those that include an epitope, in particular a B cell epitope or T cell epitope.

A B-cell epitope is conveniently derived from the amino acid sequence of an immunogenic BSX protein. Idiotypic and anti-idiotypic B cell epitopes against which an immune response is desired are specifically encompassed by the invention, as are lipid-modified B cell epitopes or a Group B protein. A preferred B-cell epitope will be capable of eliciting the production of antibodies when administered to a mammal, preferably neutralizing antibody against M. tuberculosis, and more preferably, a high titer neutralizing antibody. Shorter B cell epitopes are preferred, to facilitate peptide synthesis. Preferably, the length of the B cell epitope will not exceed about 30 amino acids in length. More preferably, the B cell epitope sequence consists of about 25 amino acid residues or less, and more preferably less than 20 amino acid residues, and even more preferably about 5-20 amino acid residues in length derived from the sequence of the full-length Group B protein.

A CTL epitope is also conveniently derived from the full length amino acid sequence of a BSX protein and will generally consist of at least about 9 contiguous amino acids of said BSX protein and have an amino acid sequence that interacts at a significant level with a MHC Class I allele as determined using a predictive algorithm for determining MHC Class I-binding epitopes, such as, for example, the SYFPEITHI algorithm of the University of Tuebingen, Germany, or the algorithm of the HLA Peptide Binding Predictions program of the BioInformatics and Molecular Analysis Section (BIMAS) of the National Institutes of Health of the Government of the United States of America. More preferably, the CTL epitope will have an amino acid sequence that binds to and/or stabilizes a MHC Class I molecule on the surface of an antigen presenting cell (APC). Even more preferably, the CTL epitope will have a sequence that induces a memory CTL response or elicits IFN-γ expression by a T cell, such as, for example, CD8⁺ T cell, cytotoxic T cell (CTL). Still even more preferably, the CTL will have a sequence that stimulates CTL activity in a standard cytotoxicity assay. Particularly preferred CTL epitopes of a BSX protein are capable of eliciting a cellular immune response against M. tuberculosis in human cells or tissues, such as, for example, by recognizing and lysing human cells infected with M. tuberculosis, thereby providing or enhancing cellular immunity against M. tuberculosis.

Suitable fragments will be at least about 5, e.g. 10, 12, 15 or 20 amino acids in length. They may also be less than 200, 100 or 50 amino acids in length.

Preferably, an immunogenic fragment or epitope of BSX comprises an amino acid sequence set forth in any one of SEQ ID Nos: 2-53, and preferably an immunogenic fragment or epitope thereof comprising an amino acid sequence selected from the group consisting of: MRQLAERSGVSNPYL (SEQ ID NO: 14), ERGLRKPSADVLSQI (SEQ ID NO: 20), LRKPSADVLSQIAKA (SEQ ID NO: 21), PSADVLSQIAKALRV (SEQ ID NO: 22), SQIAKALRVSAEVLY (SEQ ID NO: 24), AKALRVSAEVLYVRA (SEQ ID NO: 25), VRAGILEPSETSQVR (SEQ ID No: 29), TAITERQKQILLDIY (SEQ ID NO; 36), SQIAKALRVSAEVLYVRAC (SEQ ID NO: 45), MSSEEKLCDPTPTDD (SEQ ID NO: 46) and VRAGILEPSETSQVRC (SEQ ID NO: 47).

Still more preferably, an immunogenic fragment of a BSX protein of M. tuberculosis will comprise an amino acid sequence selected from the group consisting of: SQIAKALRVSAEVLY (SEQ ID NO: 24), AKALRVSAEVLYVRA (SEQ ID NO: 25), SQIAKALRVSAEVLYVRAC (SEQ ID NO: 45), MSSEEKLCDPTPTDD (SEQ ID NO: 46) and VRAGILEPSETSQVRC (SEQ ID NO: 47), and still more preferably a sequence selected from the group consisting of: SQIAKALRVSAEVLY (SEQ ID NO: 24), AKALRVSAEVLYVRA (SEQ ID NO: 25), and SQIAKALRVSAEVLYVRAC (SEQ ID NO: 45).

The amino acid sequence of a BSX protein or immunogenic fragment or epitope thereof may be modified for particular purposes according to methods well known to those of skill in the art without adversely affecting its immune function. For example, particular peptide residues may be derivatized or chemically modified in order to enhance the immune response or to permit coupling of the peptide to other agents, particularly lipids. It also is possible to change particular amino acids within the peptides without disturbing the overall structure or antigenicity of the peptide. Such changes are therefore termed “conservative” changes and tend to rely on the hydrophilicity or polarity of the residue. The size and/or charge of the side chains also are relevant factors in determining which substitutions are conservative.

The present invention clearly encompasses a covalent fusion between one or more immunogenic BSX peptides, including a homo-dimer, homo-trimer, homo-tetramer or higher order homo-multimer of a peptide, or a hetero-dimer, hetero-trimer, hetero-tetramer or higher order hetero-multimer comprising two or more different immunogenic peptides.

The present invention also encompasses a non-covalent aggregate between one or more immunogenic BSX peptides, e.g., held together by ionic, hydrostatic or other interaction known in the art or described herein.

It is well understood by the skilled artisan that, inherent in the definition of a biologically functional equivalent protein is the concept that there is a limit to the number of changes that may be made within a defined portion of the molecule and still result in a molecule with an acceptable level of equivalent biological activity. Biologically functional equivalent proteins are thus defined herein as those proteins in which specific amino acids are substituted. Particular embodiments encompass variants that have one, two, three, four, five or more variations in the amino acid sequence of the peptide. Of course, a plurality of distinct proteins/peptides with different substitutions may easily be made and used in accordance with the invention.

Those skilled in the art are well aware that the following substitutions are permissible conservative substitutions (i) substitutions involving arginine, lysine and histidine; (ii) substitutions involving alanine, glycine and serine; and (iii) substitutions involving phenylalanine, tryptophan and tyrosine. Derivatives incorporating such conservative substitutions are defined herein as biologically or immunologically functional equivalents.

The importance of the hydropathic amino acid index in conferring interactive biological function on a protein is generally understood in the art (Kyte & Doolittle, J. Mol. Biol. 157, 105-132, 1982). It is known that certain amino acids may be substituted for other amino acids having a similar hydropathic index or score and still retain a similar biological activity. The hydropathic index of amino acids also may be considered in determining a conservative substitution that produces a functionally equivalent molecule. Each amino acid has been assigned a hydropathic index on the basis of their hydrophobicity and charge characteristics, as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making changes based upon the hydropathic index, the substitution of amino acids whose hydropathic indices are within .+/−0.2 is preferred. More preferably, the substitution will involve amino acids having hydropathic indices within .+/−0.1, and more preferably within about +/−0.05.

It is also understood in the art that the substitution of like amino acids is made effectively on the basis of hydrophilicity, particularly where the biological functional equivalent protein or peptide thereby created is intended for use in immunological embodiments, as in the present case (e.g. U.S. Pat. No. 4,554,101), In fact, the greatest local average hydrophilicity of a protein, as governed by the hydrophilicity of its adjacent amino acids, correlates with its immunogenicity and antigenicity. As detailed in U.S. Pat. No. 4,554,101, the following hydrophilicity values have been assigned to amino acid residues: arginine (+3.0); lysine (+3.0); aspartate (+3.0+/−0.1); glutamate (+3.0+/−0.1); serine (+0.3); asparagine (+0.2); glutamine (+0.2); glycine (0); threonine (−0.4); proline (−0.5+/−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, it is preferred to substitute amino acids having hydrophilicity values within about +/−0.2 of each other, more preferably within about +/−0.1, and even more preferably within about +/−0.05

The BSX polypeptide or peptide fragment thereof comprising an epitope is readily synthesized using standard techniques, such as the Merrifield method of synthesis (Merrifield, J Am Chem Soc, 85, 2149-2154, 1963) and the myriad of available improvements on that technology (see e.g., Synthetic Peptides: A User's Guide, Grant, ed. (1992) W.H. Freeman & Co., New York, pp. 382; Jones (1994) The Chemical Synthesis of Peptides, Clarendon Press, Oxford, pp. 230); Barany, G. and Merrifield, R. B. (1979) in The Peptides (Gross, E. and Meienhofer, J. eds.), vol. 2, pp. 1-284, Academic Press, New York; Wiinsch, E., ed. (1974) Synthese von Peptiden in Houben-Weyls Metoden der Organischen Chemie (Müler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474.d/

As is known in the art, synthetic peptides can be produced with additional hydrophilic N-terminal and/or C-terminal amino acids added to the sequence of a fragment or B-cell epitope derived from the full-length BSX protein, such as, for example, to facilitate synthesis or improve peptide solubility. Glycine and/or serine residues are particularly preferred for this purpose. As exemplified herein, each of the peptides set forth in SEQ ID NOs 2-44 includes additional spacer sequences flanking the BSX fragments, said spacers comprising hetero-polymers (trimers or tetramers) comprising glycine and serine.

The peptides of the invention are readily modified for diagnostic purposes, for example, by addition of a natural or synthetic hapten, an antibiotic, hormone, steroid, nucleoside, nucleotide, nucleic acid, an enzyme, enzyme substrate, an enzyme inhibitor, biotin, avidin, streptavidin, polyethylene glycol, a peptidic polypeptide moiety (e.g. tuftsin, poly-lysine), a fluorescence marker (e.g. FITC, RITC, dansyl, luminol or coumarin), a bioluminescence marker, a spin label, an alkaloid, biogenic amine, vitamin, toxin (e.g. digoxin, phalloidin, amanitin, tetrodotoxin), or a complex-forming agent.

In another embodiment, a BSX protein is produced as a recombinant protein.

For expressing protein by recombinant means, a protein-encoding nucleotide sequence is placed in operable connection with a promoter or other regulatory sequence capable of regulating expression in a cell-free system or cellular system. In one embodiment of the invention, nucleic acid comprising a sequence that encodes a BSX protein or an epitope thereof in operable connection with a suitable promoter sequence, is expressed in a suitable cell for a time and under conditions sufficient for expression to occur. Nucleic acid encoding the BSX protein is readily derived from the publicly available amino acid sequence.

In another embodiment, a BSX protein is produced as a recombinant fusion protein, such as for example, to aid in extraction and purification. To produce a fusion polypeptide, the open reading frames are covalently linked in the same reading frame, such as, for example, using standard cloning procedures as described by Ausubel et al. (Current Protocols in Molecular Biology, Wiley Interscience, ISBN 047150338, 1992), and expressed under control of a promoter. Examples of fusion protein partners include glutathione-S-transferase (GST), FLAG (Asp-Tyr-Lys-Asp-Asp-Asp-Asp-Lys), hexa-histidine, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the immune function of the BSX protein.

Reference herein to a “promoter” is to be taken in its broadest context and includes the transcriptional regulatory sequences of a classical genomic gene, including the TATA box which is required for accurate transcription initiation, with or without a CCAAT box sequence and additional regulatory elements (i.e., upstream activating sequences, enhancers and silencers) which alter gene expression in response to developmental and/or external stimuli, or in a tissue-specific manner. In the present context, the term “promoter” is also used to describe a recombinant, synthetic or fusion molecule, or derivative which confers, activates or enhances the expression of a nucleic acid molecule to which it is operably connected, and which encodes the polypeptide or peptide fragment. Preferred promoters can contain additional copies of one or more specific regulatory elements to further enhance expression and/or to alter the spatial expression and/or temporal expression of the said nucleic acid molecule.

Placing a nucleic acid molecule under the regulatory control of, i.e., “in operable connection with”, a promoter sequence means positioning said molecule such that expression is controlled by the promoter sequence. Promoters are generally positioned 5′ (upstream) to the coding sequence that they control.

The prerequisite for producing intact polypeptides and peptides in bacteria such as E. coli is the use of a strong promoter with an effective ribosome binding site. Typical promoters suitable for expression in bacterial cells such as E. coli include, but are not limited to, the lacz promoter, temperature-sensitive λ_{L} or λ_{R} promoters, T7 promoter or the IPTG-inducible tac promoter. A number of other vector systems for expressing the nucleic acid molecule of the invention in E. coli are well-known in the art and are described, for example, in Ausubel et al (In: Current Protocols in Molecular Biology. Wiley Interscience, ISBN 047150338, 1987) or Sambrook et al (In: Molecular cloning, A laboratory manual, second edition, Cold Spring Harbor Laboratory, Cold Spring Harbor, N.Y., 1989). Numerous plasmids with suitable promoter sequences for expression in bacteria and efficient ribosome binding sites have been described, such as for example, pKC30 (λ_{L}: Shimatake and Rosenberg, Nature 292, 128, 1981); p 173-3 (tac: Amann and Brosius, Gene 40, 183, 1985), pET-3 (T7: Studier and Moffat, J. Mol. Biol. 189, 113, 1986); the pBAD/TOPO or pBAD/Thio-TOPO series of vectors containing an arabinose-inducible promoter (Invitrogen, Carlsbad, Calif.), the latter of which is designed to also produce fusion proteins with thioredoxin to enhance solubility of the expressed protein; the pFLEX series of expression vectors (Pfizer Inc., CT, USA); or the pQE series of expression vectors (Qiagen, CA), amongst others.

Typical promoters suitable for expression in viruses of eukaryotic cells and eukaryotic cells include the SV40 late promoter, SV40 early promoter and cytomegalovirus (CMV) promoter, CMV IE (cytomegalovirus immediate early) promoter amongst others. Preferred vectors for expression in mammalian cells (e.g. 293, COS, CHO, 10T cells, 293T cells) include, but are not limited to, the pcDNA vector suite supplied by Invitrogen, in particular pcDNA 3.1 myc-His-tag comprising the CMV promoter and encoding a C-terminal 6×His and MYC tag; and the retrovirus vector pSRαtkneo (Muller et al., Mol. Cell. Biol., 11, 1785, 1991). The vector pcDNA 3.1 myc-His (Invitrogen) is particularly preferred for expressing a secreted form of a BSX protein or a derivative thereof in 293T cells, wherein the expressed peptide or protein can be purified free of conspecific proteins, using standard affinity techniques that employ a Nickel column to bind the protein via the His tag.

A wide range of additional host/vector systems suitable for expressing the diagnostic protein of the present invention or an immunological derivative (e.g., an epitope or other fragment) thereof are available publicly, and described, for example, in Sambrook et al (In: Molecular cloning, A laboratory manual, second edition, Cold Spring Harbor Laboratory, Cold Spring Harbor, N.Y., 1989).

Means for introducing the isolated nucleic acid molecule or a gene construct comprising same into a cell for expression are well-known to those skilled in the art. The technique used for a given organism depends on the known successful techniques. Means for introducing recombinant DNA into animal cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

Proteins of the invention can be produced in an isolated form, preferably substantially free of conspecific protein. Antibodies and other affinity ligands are particularly preferred for producing isolated protein. Preferably, the protein will be in a preparation wherein more than about 90% (e.g. 95%, 98% or 99%) of the protein in the preparation is a BSX protein or an epitope thereof.

Antibodies that Bind to a BSX Protein or an Epitope Thereof

A second aspect of the present invention provides an antibody that binds specifically to a BSX protein or an immunogenic fragment or epitope thereof, such as, for example, a monoclonal or polyclonal antibody preparation suitable for use in the assays described herein.

Reference herein to antibody or antibodies includes whole polyclonal and monoclonal antibodies, and parts thereof, either alone or conjugated with other moieties. Antibody parts include Fab and F(ab)₂ fragments and single chain antibodies. The antibodies may be made in vivo in suitable laboratory animals, or, in the case of engineered antibodies (Single Chain Antibodies or SCABS, etc) using recombinant DNA techniques in vitro.

In accordance with this aspect of the invention, the antibodies may be produced for the purposes of immunizing the subject, in which case high titer or neutralizing antibodies that bind to a B cell epitope will be especially preferred. Suitable subjects for immunization will, of course, depend upon the immunizing antigen or antigenic B cell epitope. It is contemplated that the present invention will be broadly applicable to the immunization of a wide range of animals, such as, for example, farm animals (e.g. horses, cattle, sheep, pigs, goats, chickens, ducks, turkeys, and the like), laboratory animals (e.g. rats, mice, guinea pigs, rabbits), domestic animals (cats, dogs, birds and the like), feral or wild exotic animals (e.g. possums, cats, pigs, buffalo, wild dogs and the like) and humans.

Alternatively, the antibodies may be for commercial or diagnostic purposes, in which case the subject to whom the BSX protein or immunogenic fragment or epitope thereof is administered will most likely be a laboratory or farm animal. A wide range of animal species are used for the production of antisera. Typically the animal used for production of antisera is a rabbit, mouse, rabbit, rat, hamster, guinea pig, goat, sheep, pig, dog, horse, or chicken. Because of the relatively large blood volumes of rabbits and sheep, these are preferred choice for production of polyclonal antibodies. However, as will be known to those skilled in the art, larger amounts of immunogen are required to obtain high antibodies from large animals as opposed to smaller animals such as mice. In such cases, it will be desirable to isolate the antibody from the immunized animal.

Preferably, the antibody is a high titer antibody. By “high titer” means a sufficiently high titer to be suitable for use in diagnostic or therapeutic applications. As will be known in the art, there is some variation in what might be considered “high titer”. For most applications a titer of at least about 10³-10⁴ is preferred. More preferably, the antibody titer will be in the range from about 10⁴ to about 10⁵, even more preferably in the range from about 10⁵ to about 10⁶.

More preferably, in the case of B cell epitopes from pathogens, viruses or bacteria, the antibody is a neutralizing antibody (i.e. it is capable of neutralizing the infectivity of the organism fro which the B cell epitope is derived).

To generate antibodies, the BSX protein or immunogenic fragment or epitope thereof, optionally formulated with any suitable or desired carrier, adjuvant, BRM, or pharmaceutically acceptable excipient, is conveniently administered in the form of an injectable composition. Injection may be intranasal, intramuscular, sub-cutaneous, intravenous, intradermal, intraperitoneal, or by other known route. For intravenous injection, it is desirable to include one or more fluid and nutrient replenishers. Means for preparing and characterizing antibodies are well known in the art. (See, e.g., ANTIBODIES: A LABORATORY MANUAL, Cold Spring Harbor Laboratory, 1988, incorporated herein by reference).

Preferred immunogenic peptides for generating polyclonal or monoclonal antibodies are selected from the group set forth in the Sequence Listing. In one embodiment, an immunogenic peptide such as, for example, an immunogenic peptide comprising the amino acid sequence set forth in SEQ ID NO: 24, 25 45 or 46 or an immunogenic fragment thereof, is covalently coupled to an immunogenic carrier protein, such as Diphtheria toxoid (DT), Keyhole Limpet Hemocyanin (KLH), tetanus toxoid (TT) or the nuclear protein of influenza virus (NP), using one of several conjugation chemistries known in the art. This enhances the immunogenicity of peptides that are otherwise not highly immunogenic in animals e.g., mice, rats, chickens etc.

Methods of preparing carrier proteins for such coupling are well known in the art. For instance, DT is preferably produced by purification of the toxin from a culture of Corynebacterium diphtheriae followed by chemical detoxification, but is alternatively made by purification of a recombinant, or genetically detoxified analogue of the toxin (for example, CRM197, or other mutants as described in U.S. Pat. Nos. 4,709,017, 5,843,711, 5,601,827, and 5,917,017). Preferably, the toxoid is derivatized using as a spacer a bridge of up to 6 carbons, such as provided by use of the adipic acid hydrazide derivative of diphtheria toxoid (D-AH).

For coupling, peptides derived from the full-length BSX protein can be synthesized chemically or produced by recombinant expression means, treated with hydroxylamine to form free sulfhydryl groups, and cross-linked via the free sulfhydryl groups to a maleimide-modified diphtheria toxoid, tetanus toxoid or influenza NP protein or other carrier molecule. One of the most specific and reliable conjugation chemistries uses a cysteine residue in the peptide and a maleimide group added to the carrier protein, to form a stable thioether bond (Lee, A. C., et al., Mol. Immunol. 17, 749-756 1980). For example, if no sulfhydryl groups are present in the peptide, the BSX-derived peptides can be prior modified by the addition of a C-terminal cysteine residue e.g., SEQ ID NO: 45, or an internal cysteine residue, e.g., SEQ ID NO: 46, to facilitate this procedure. The immunogenic BSX peptides are preferably produced under non-denaturing conditions, treated with hydroxylamine, thiol reducing agents or by acid or base hydrolysis to generate free sulfhydryl groups and the free sulfhydryl-containing peptide is conjugated to a carrier by chemical bonding via the free sulfhydryl groups. Such conjugation may be by use of a suitable bis-maleimide compound. Alternatively, the conjugation of the HA protein may be to a maleimide-modified carrier protein, such as diphtheria toxoid, tetanus toxoid or influenza (NP) protein or to a carbohydrate, such as alginic acid, dextran or polyethylene glycol. Such maleimide-modified carrier molecules may be formed by reaction of the carrier molecule with a hetero-bifunctional cross-linker of the maleimide-N-hydroxysuccinimide ester type. Examples of such bifunctional esters include maleimido-caproic-N-hydroxysuccinimide ester (MCS), maleimido-benzoyl-N-hydroxysuccinimide ester (MBS), maleimido-benzoylsulfosuccinimide ester (sulfo-MBS), succinimidyl-4-(N-maleimidomethyl)cyclohexane-1-carboxylate (SMCC), succinimidyl-4-(p-maleimido-phenyl)butyrate (SMPP), sulfosuccinimidyl-4-(N-maleimidomethyl)cyclohexane-1-carboxylate (sulfo-SMCC) and sulfosuccinimidyl-4-(p-maleimidophenyl)butyrate (sulfo-SMPP). The N-hydroxy-succinimide ester moiety reacts with the amine groups of the carrier protein leaving the maleimide moiety free to react with the sulfhydryl groups on the antigen to form the cross-linked material.

The conjugate molecules so produced may be purified and employed in immunogenic compositions to elicit, upon administration to a host, an immune response to the BSX peptide which is potentiated in comparison to BSX peptide alone.

Diphtheria toxoid is obtained commercially or prepared from Corynebacterium diphtheriae grown in submerged culture by standard methods. The production of Diphtheria Toxoid is divided into five stages, namely maintenance of the working seed, growth of Corynebacterium diphtheriae, harvest of Diphtheria Toxin, detoxification of Diphtheria Toxin and concentration of Diphtheria Toxoid. The purified diphtheria toxoid (DT) used as carrier in the preparation is preferably a commercial toxoid modified (derivatized) by the attachment of a spacer molecule, such as adipic acid dihydrazide (ADH), using the water-soluble carbodiimide condensation method. The modified toxoid, typically the adipic hydrazide derivative D-AH, is then freed from unreacted ADH.

The efficacy of the BSX protein or immunogenic fragment or epitope thereof in producing an antibody is established by injecting an animal, for example, a mouse, chicken, rat, rabbit, guinea pig, dog, horse, cow, goat or pig, with a formulation comprising the BSX protein or immunogenic fragment or epitope thereof, and then monitoring the immune response to the B cell epitope, as described in the Examples. Both primary and secondary immune responses are monitored. The antibody titer is determined using any conventional immunoassay, such as, for example, ELISA, or radio immunoassay.

The production of polyclonal antibodies may be monitored by sampling blood of the immunized animal at various points following immunization. A second, booster injection, may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies (Mabs).

Monoclonal antibodies are particularly preferred. For the production of monoclonal antibodies (Mabs) any one of a number of well-known techniques may be used, such as, for example, the procedure exemplified in U.S. Pat. No. 4,196,265, incorporated herein by reference.

For example, a suitable animal will be immunized with an effective amount of the BSX protein or immunogenic fragment or epitope thereof under conditions sufficient to stimulate antibody producing cells. Rodents such as rabbits, mice and rats are preferred animals, however, the use of sheep or frog cells is also possible. The use of rats may provide certain advantages, but mice or rabbits are preferred, with the BALB/c mouse being most preferred as the most routinely used animal and one that generally gives a higher percentage of stable fusions. Rabbits are known to provide high affinity monoclonal antibodies.

Following immunization, somatic cells with the potential for producing antibodies, specifically B lymphocytes (B cells), are selected for use in the MAb generating protocol. These cells may be obtained from biopsies of spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer removed. Spleen lymphocytes are obtained by homogenizing the spleen with a syringe. Typically, a spleen from an immunized mouse contains approximately 5×10⁷ to 2×10⁸ lymphocytes.

The B cells from the immunized animal are then fused with cells of an immortal myeloma cell, generally derived from the same species as the animal that was immunized with the BSX protein or immunogenic fragment or epitope thereof. Myeloma cell lines suited for use in hybridoma-producing fusion procedures preferably are non-antibody-producing, have high fusion efficiency and enzyme deficiencies that render them incapable of growing in certain selective media which support the growth of only the desired fused cells, or hybridomas. Any one of a number of myeloma cells may be used and these are known to those of skill in the art (e.g. murine P3-X63/Ag8, X63-Ag8.653, NS1/1.Ag 4 1, Sp210-Ag14, FO, NSO/U, MPC-11, MPC11-X45-GTG 1.7 and S194/5XX0; or rat R210.RCY3, Y3-Ag 1.2.3, IR983F and 4B210; and U-266, GM1500-GRG2, LICR-LON-HMy2 and UC729-6). A preferred murine myeloma cell is the NS-1 myeloma cell line (also termed P3-NS-1-Ag4-1), which is readily available from the NIGMS Human Genetic Mutant Cell Repository under Accession No. GM3573. Alternatively, a murine myeloma SP2/0 non-producer cell line that is 8-azaguanine-resistant is used.

To generate hybrids of antibody-producing spleen or lymph node cells and myeloma cells, somatic cells are mixed with myeloma cells in a proportion between about 20:1 to about 1:1, respectively, in the presence of an agent or agents (chemical or electrical) that promote the fusion of cell membranes. Fusion methods using Sendai virus have been described by Kohler and Milstein, Nature 256, 495-497, 1975; and Kohler and Milstein, Eur. J. Immunol. 6, 511-519, 1976. Methods using polyethylene glycol (PEG), such as 37% (v/v) PEG, are described in detail by Gefter et al., Somatic Cell Genet. 3, 231-236, 1977. The use of electrically induced fusion methods is also appropriate.

Hybrids are amplified by culture in a selective medium comprising an agent that blocks the de novo synthesis of nucleotides in the tissue culture media. Exemplary and preferred agents are aminopterin, methotrexate and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT, because only those hybridomas capable of operating nucleotide salvage pathways are able to survive in HAT medium, whereas myeloma cells are defective in key enzymes of the salvage pathway, (e.g., hypoxanthine phosphoribosyl transferase or HPRT), and they cannot survive. B cells can operate this salvage pathway, but they have a limited life span in culture and generally die within about two weeks. Accordingly, the only cells that can survive in the selective media are those hybrids formed from myeloma and B cells.

The amplified hybridomas are subjected to a functional selection for antibody specificity and/or titer, such as, for example, by immunoassay (e.g. radioimmunoassay, enzyme immunoassay, cytotoxicity assay, plaque assay, dot immunoassay, and the like).

The selected hybridomas are serially diluted and cloned into individual antibody-producing cell lines, which clones can then be propagated indefinitely to provide MAbs. The cell lines may be exploited for MAb production in two basic ways. A sample of the hybridoma is injected, usually in the peritoneal cavity, into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they are readily obtained in high concentrations. MAbs produced by either means may be further purified, if desired, using filtration, centrifugation and various chromatographic methods such as HPLC or affinity chromatography.

Alternatively, ABL-MYC technology (NeoClone, Madison Wis. 53713, USA) is used to produce cell lines secreting monoclonal antibodies (mAbs) against immunogenic BSX peptide antigens. In this process, BALB/cByJ female mice are immunized with an amount of the peptide antigen over a period of about 2 to about 3 months. During this time, test bleeds are taken from the immunized mice at regular intervals to assess antibody responses in a standard ELISA. The spleens of mice having antibody titers of at least about 1,000 are used for subsequent ABL-MYC infection employing replication-incompetent retrovirus comprising the oncogenes v-abl and c-myc. Splenocytes are transplanted into naive mice which then develop ascites fluid containing cell lines producing monoclonal antibodies (mAbs) against the BSX peptide antigen. The mAbs are purified from ascites using protein G or protein A, e.g., bound to a solid matrix, depending on the isotype of the mAb. Because there is no hybridoma fusion, an advantage of the ABL-MYC process is that it is faster, more cost effective, and higher yielding than conventional mAb production methods. In addition, the diploid plasmacytomas produced by this method are intrinsically more stable than polyploid hybridomas, because the ABL-MYC retrovirus infects only cells in the spleen that have been stimulated by the immunizing antigen. ABL-MYC then transforms those activated B-cells into immortal, mAb-producing plasma cells called plasmacytomas. A “plasmacytoma” is an immortalized plasma cell that is capable of uncontrolled cell division. Since a plasmacytoma begins with just one cell, all of the plasmacytomas produced from it are therefore identical, and moreover, produce the same desired “monoclonal” antibody. As a result, no sorting of undesirable cell lines is required. The ABL-MYC technology is described generically in detail in the following disclosures which are incorporated by reference herein:

1. Largaespada et al., Curr. Top. Microbiol. Immunol., 166, 91-96. 1990;
2. Weissinger et al., Proc. Natl. Acad. Sci. USA, 88, 8735-8739, 1991;

### 3. Largaespada et al., Oncogene, 7, 811-819, 1992;

4. Weissinger et al., J. Immunol. Methods 168, 123-130, 1994;
5. Largaespada et al., J. Immunol. Methods. 197(1-2), 85-95, 1996; and
6. Kumar et al., Immuno. Letters 65, 153-159, 1999.

Monoclonal antibodies of the present invention also include anti-idiotypic antibodies produced by methods well-known in the art. Monoclonal antibodies according to the present invention also may be monoclonal heteroconjugates, (i.e., hybrids of two or more antibody molecules). In another embodiment, monoclonal antibodies according to the invention are chimeric monoclonal antibodies. In one approach, the chimeric monoclonal antibody is engineered by cloning recombinant DNA containing the promoter, leader, and variable-region sequences from a mouse anti-PSA producing cell and the constant-region exons from a human antibody gene. The antibody encoded by such a recombinant gene is a mouse-human chimera. Its antibody specificity is determined by the variable region derived from mouse sequences. Its isotype, which is determined by the constant region, is derived from human DNA.

In another embodiment, the monoclonal antibody according to the present invention is a “humanized” monoclonal antibody, produced by any one of a number of techniques well-known in the art. That is, mouse complementary determining regions (“CDRs”) are transferred from heavy and light V-chains of the mouse Ig into a human V-domain, followed by the replacement of some human residues in the framework regions of their murine counterparts. “Humanized” monoclonal antibodies in accordance with this invention are especially suitable for use in vivo in diagnostic and therapeutic methods.

As stated above, the monoclonal antibodies and fragments thereof according to this invention are multiplied according to in vitro and in vivo methods well-known in the art. Multiplication in vitro is carried out in suitable culture media such as Dulbecco's modified Eagle medium or RPMI 1640 medium, optionally replenished by a mammalian serum such as fetal calf serum or trace elements and growth-sustaining supplements, e.g., feeder cells, such as normal mouse peritoneal exudate cells, spleen cells, bone marrow macrophages or the like. In vitro production provides relatively pure antibody preparations and allows scale-up to give large amounts of the desired antibodies. Techniques for large scale hybridoma cultivation under tissue culture conditions are known in the art and include homogenous suspension culture, (e.g., in an airlift reactor or in a continuous stirrer reactor or immobilized or entrapped cell culture).

Large amounts of the monoclonal antibody of the present invention also may be obtained by multiplying hybridoma cells in vivo. Cell clones are injected into mammals which are histocompatible with the parent cells, (e.g., syngeneic mice, to cause growth of antibody-producing tumors. Optionally, the animals are primed with a hydrocarbon, especially oils such as Pristane (tetramethylpentadecane) prior to injection.

In accordance with the present invention, fragments of the monoclonal antibody of the invention are obtained from monoclonal antibodies produced as described above, by methods which include digestion with enzymes such as pepsin or papain and/or cleavage of disulfide bonds by chemical reduction. Alternatively, monoclonal antibody fragments encompassed by the present invention are synthesized using an automated peptide synthesizer, or they may be produced manually using techniques well known in the art.

The monoclonal conjugates of the present invention are prepared by methods known in the art, e.g., by reacting a monoclonal antibody prepared as described above with, for instance, an enzyme in the presence of a coupling agent such as glutaraldehyde or periodate. Conjugates with fluorescein markers are prepared in the presence of these coupling agents, or by reaction with an isothiocyanate. Conjugates with metal chelates are similarly produced. Other moieties to which antibodies may be conjugated include radionuclides such as, for example, ³H, ¹²⁵I, ³²P, ³⁵S, ¹⁴C, ⁵¹Cr, ³⁶Cl, ⁵⁷Co, ⁵⁸Co, ⁵⁹Fe, ⁷⁵Se, and ¹⁵²Eu.

Radioactively labelled monoclonal antibodies of the present invention are produced according to well-known methods in the art. For instance, monoclonal antibodies are iodinated by contact with sodium or potassium iodide and a chemical oxidizing agent such as sodium hypochlorite, or an enzymatic oxidizing agent, such as lactoperoxidase. Monoclonal antibodies according to the invention may be labelled with technetium⁹⁹ by ligand exchange process, for example, by reducing pertechnate with stannous solution, chelating the reduced technetium onto a Sephadex column and applying the antibody to this column or by direct labelling techniques, (e.g., by incubating pertechnate, a reducing agent such as SNCl₂, a buffer solution such as sodium-potassium phthalate solution, and the antibody).

Any immunoassay may be used to monitor antibody production by the BSX protein or immunogenic fragment or epitope thereof. Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and Western blotting, dot blotting, FACS analyses, and the like may also be used.

Most preferably, the assay will be capable of generating quantitative results.

For example, antibodies are tested in simple competition assays. A known antibody preparation that binds to the B cell epitope and the test antibody are incubated with an antigen composition comprising the B cell epitope, preferably in the context of the native antigen. “Antigen composition” as used herein means any composition that contains some version of the B cell epitope in an accessible form. Antigen-coated wells of an ELISA plate are particularly preferred. In one embodiment, one would pre-mix the known antibodies with varying amounts of the test antibodies (e.g., 1:1, 1:10 and 1:100) for a period of time prior to applying to the antigen composition. If one of the known antibodies is labelled, direct detection of the label bound to the antigen is possible; comparison to an unmixed sample assay will determine competition by the test antibody and, hence, cross-reactivity. Alternatively, using secondary antibodies specific for either the known or test antibody, one will be able to determine competition.

An antibody that binds to the antigen composition will be able to effectively compete for binding of the known antibody and thus will significantly reduce binding of the latter. The reactivity of the known antibodies in the absence of any test antibody is the control. A significant reduction in reactivity in the presence of a test antibody is indicative of a test antibody that binds to the B cell epitope (i.e., it cross-reacts with the known antibody).

In one exemplary ELISA, the antibodies against the BSX protein or immunogenic fragment or B cell epitope are immobilized onto a selected surface exhibiting protein affinity, such as a well in a polystyrene microtiter plate. Then, a composition containing a peptide comprising the B cell epitope is added to the wells. After binding and washing to remove non-specifically bound immune complexes, the bound epitope may be detected. Detection is generally achieved by the addition of a second antibody that is known to bind to the B cell epitope and is linked to a detectable label. This type of ELISA is a simple “sandwich ELISA”. Detection may also be achieved by the addition of said second antibody, followed by the addition of a third antibody that has binding affinity for the second antibody, with the third antibody being linked to a detectable label.

In another exemplary immunoassay format applicable to both flow through and solid phase ELISA, antibodies that bind to the immunogenic BSX protein or immunogenic BSX peptide or immunogenic BSX fragment or B cell epitope are immobilized onto a selected surface exhibiting protein affinity, such as a well in a polystyrene microtiter plate or a column. A sample comprising the immunogenic BSX protein or immunogenic peptide or immunogenic fragment comprising the B cell epitope to which the antibody binds is added for a time and under conditions sufficient for an antigen-antibody complex to form. In this case, the added BSX protein, peptide or fragment is complexed with human Ig. In the case of patient sera, for example, the peptide is preferably complexed with human Ig by virtue of an immune response of the patient to the M. tuberculosis BSX protein. After binding and washing to remove non-specifically bound immune complexes, the bound epitope is detected by the addition of a second antibody that is known to bind to human Ig in the immune complex and is linked to a detectable label. This is a modified “sandwich ELISA”. Detection may also be achieved by the addition of said second antibody, followed by the addition of a third antibody that has binding affinity for the second antibody, with the third antibody being linked to a detectable label.

The present invention clearly encompasses multianalyte tests for diagnosing infection by M. tuberculosis. For example, assays for detecting antibodies against M. tuberculosis BSX protein can be combined with assays for detecting antibodies against M. tuberculosis glutamine synthetase (GS). In this respect, the present inventors have also produced a plasmacytoma producing monoclonal antibodies that bind to an immunogenic fragment or peptide or epitope of GS that comprises an amino acid sequence of at least about 5 consecutive amino acid residues positioned from about residue 265 to about residue 300 of full-length GS, more preferably from about residue 270 to about residue 295 of GS and still more preferably from residue 271 to residue 295 of GS, and especially to at least 5 consecutive residues within the amino acid sequence RGTDGSAVFADSNGPHGMSSMFRSF (SEQ ID NO: 54). Thus, the antibodies bind to 5 or 6 or 7 or 8 or 9 or 10 or 11 or 12 or 13 or 14 or 15 or 16 or 17 or 18 or 19 or 20 or 21 or 22 or 23 or 24 or 25 consecutive amino acid residues of SEQ ID NO: 54 further comprising N-terminal and C-terminal extensions not adversely affecting immunogenicity of the base peptide).

Antibodies of the invention may be bound to a solid support and/or packaged into kits in a suitable container along with suitable reagents, controls, instructions and the like.

### Diagnostic/Prognostic Methods for Detecting Tuberculosis or M. Tuberculosis Infection

### 1. Antigen-Based Assays

This invention provides a method of diagnosing tuberculosis or an infection by M. tuberculosis in a subject comprising detecting in a biological sample from said subject a BSX protein or an immunogenic fragment or epitope thereof, wherein the presence of said protein or immunogenic fragment or epitope in the sample is indicative of infection.

One advantage of detecting M. tuberculosis antigen, as opposed to an antibody-based assay is that severely immunocompromised patients may not produce antibody at detectable levels, and the level of the antibody in any patient does not reflect bacilli burden. On the other hand antigen levels should reflect bacilli burden and, being a product of the bacilli, are a direct method of detecting its presence.

In one embodiment of the diagnostic assays of the invention, there is provided a method for detecting M. tuberculosis infection in a subject, the method comprising contacting a biological sample derived from the subject with an antibody capable of binding to a BSX protein or an immunogenic fragment or epitope thereof, and detecting the formation of an antigen-antibody complex.

In another embodiment, the diagnostic assays of the invention are useful for determining the progression of tuberculosis or an infection by M. tuberculosis in a subject. In accordance with these prognostic applications of the invention, the level of BSX protein or an immunogenic fragment or epitope thereof in a biological sample is positively correlated with the infectious state. For example, a level of the BSX protein or an immunogenic fragment thereof that is less than the level of the BSX protein or fragment detectable in a subject suffering from the symptoms of tuberculosis or an infection indicates that the subject is recovering from the infection. Similarly, a higher level of the protein or fragment in a sample from the subject compared to a healthy individual indicates that the subject has not been rendered free of the disease or infection.

Accordingly, a further embodiment of the present invention provides a method for determining the response of a subject having tuberculosis or an infection by M. tuberculosis to treatment with a therapeutic compound for said tuberculosis or infection, said method comprising detecting a BSX protein or an immunogenic fragment or epitope thereof in a biological sample from said subject, wherein a level of the protein or fragment or epitope that is enhanced compared to the level of that protein or fragment or epitope detectable in a normal or healthy subject indicates that the subject is not responding to said treatment or has not been rendered free of disease or infection.

In an alternative embodiment, the present invention provides a method for determining the response of a subject having tuberculosis or an infection by M. tuberculosis to treatment with a therapeutic compound for said tuberculosis or infection, said method comprising detecting a BSX protein or an immunogenic fragment or epitope thereof in a biological sample from said subject, wherein a level of the protein or fragment or epitope that is lower than the level of the protein or fragment or epitope detectable in a subject suffering from tuberculosis or infection by M. tuberculosis indicates that the subject is responding to said treatment or has been rendered free of disease or infection. Clearly, if the level of the BSX protein or fragment or epitope thereof is not detectable in the subject, the subject has responded to treatment.

In a further embodiment, the amount of a BSX protein in a biological sample derived from a patient is compared to the amount of the same protein detected in a biological sample previously derived from the same patient. As will be apparent to a person skilled in the art, this method may be used to continually monitor a patient with a latent infection or a patient that has developed tuberculosis. In this way a patient may be monitored for the onset or progression of an infection or disease, with the goal of commencing treatment before an infection is established, particularly in an HIV+ individual.

Alternatively, or in addition, the amount of a protein detected in a biological sample derived from a subject with tuberculosis may be compared to a reference sample, wherein the reference sample is derived from one or more tuberculosis patients that do not suffer from an infection or disease or alternatively, one or more tuberculosis patients that have recently received successful treatment for infection and/or one or more subjects that do not have tuberculosis and that do not suffer from an infection or disease.

In one embodiment, a BSX protein or immunogenic fragment thereof is not detected in a reference sample, however, said BSX protein or immunogenic fragment thereof is detected in the patient sample, indicating that the patient from whom the sample was derived is suffering from tuberculosis or infection by M. tuberculosis or will develop an acute infection.

Alternatively, the amount of BSX protein or immunogenic fragment thereof may be enhanced in the patient sample compared to the level detected in a reference sample. Again, this indicates that the patient from whom the biological sample was isolated is suffering from tuberculosis or infection by M. tuberculosis or will develop an acute infection.

In one embodiment of the diagnostic/prognostic methods described herein, the biological sample is obtained previously from the subject. In accordance with such an embodiment, the prognostic or diagnostic method is performed ex vivo.

In yet another embodiment, the subject diagnostic/prognostic methods further comprise processing the sample from the subject to produce a derivative or extract that comprises the analyte (e.g., pleural fluid or sputum).

Suitable samples include extracts from tissues such as brain, breast, ovary, lung, colon, pancreas, testes, liver, muscle and bone tissues, or body fluids such as sputum, serum, plasma, whole blood, sera or pleural fluid.

Preferably, the biological sample is a bodily fluid or tissue sample selected from the group consisting of: saliva, plasma, blood, serum, sputum, urine, and lung. Other samples are not excluded.

It will be apparent from the description herein that preferred samples may comprise circulating immune complexes comprising the BSX protein or fragments thereof complexed with human immunoglobulin. The detection of such immune complexes is clearly within the scope of the present invention. In accordance with this embodiment, a capture reagent e.g., a capture antibody is used to capture the BSX antigen (BSX polypeptide or an immunoactive fragment or epitope thereof) complexed with the subject's immunoglobulin, in addition to isolated antigen in the subject's circulation. Anti-Ig antibodies, optionally conjugated to a detectable label, are used to specifically bind the captured CIC thereby detecting CIC patient samples. Within the scope of this invention, the anti-Ig antibody binds preferentially to IgM, IgA or IgG in the sample. In a particularly preferred embodiment, the anti-Ig antibody binds to human Ig, e.g., human IgA, human IgG or human IgM. The anti-Ig antibody may be conjugated to any standard detectable label known in the art. This is particularly useful for detecting infection by a pathogenic agent, e.g., a bacterium or virus, or for the diagnosis of any disease or disorder associated with CICs. Accordingly, the diagnostic methods described according to any embodiment herein are amenable to a modification wherein the sample derived from the subject comprises one or more circulating immune complexes comprising immunoglobulin (Ig) bound to BSX protein of Mycobacterium tuberculosis or one or more immunogenic BSX peptides, fragments or epitopes thereof and wherein detecting the formation of an antigen-antibody complex comprises contacting an anti-Ig antibody with an immunoglobulin moiety of the circulating immune complex(es) for a time and under conditions sufficient for a complex to form than then detecting the bound anti-Ig antibody.

### 2. Antibody-Based Assays

The present invention provides a method of diagnosing tuberculosis or an infection by M. tuberculosis in a subject comprising detecting in a biological sample from said subject antibodies against a BSX protein or an immunogenic fragment or epitope thereof, wherein the presence of said antibodies in the sample is indicative of infection. The infection may be a past or present infection, or a latent infection.

Antibody-based assays are primarily used for detecting active infections by M. tuberculosis. Preferably, the clinical history of the subject is considered due to residual antibody levels that may persist in recent past infections or chronic infections by M. tuberculosis.

The format is inexpensive and highly sensitive, however not as useful as an antigen-based assay format for detecting infection in immunocompromised individuals. However, antibody-based assays are clearly useful for detecting M. tuberculosis infections in HIV⁻ or HIV⁺ individuals who are not immunocompromised.

In one alternative embodiment, the present invention provides a method for detecting M. tuberculosis infection in a subject, the method comprising contacting a biological sample derived from the subject with a BSX protein or an immunogenic fragment or epitope thereof and detecting the formation of an antigen-antibody complex.

In another embodiment, the diagnostic assays of the invention are useful for determining the progression of tuberculosis or an infection by M. tuberculosis in a subject. In accordance with these prognostic applications of the invention, the amount of antibodies against a BSX protein or fragment or epitope in blood or serum or an immunoglobulin fraction from the subject is positively correlated with the infectious state. For example, a level of the anti-BSX antibodies thereto that is less than the level of the anti-BSX antibodies detectable in a subject suffering from the symptoms of tuberculosis or an infection indicates that the subject is recovering from the infection. Similarly, a higher level of the antibodies in a sample from the subject compared to a healthy individual indicates that the subject has not been rendered free of the disease or infection.

In a further embodiment of the present invention provides a method for determining the response of a subject having tuberculosis or an infection by M. tuberculosis to treatment with a therapeutic compound for said tuberculosis or infection, said method comprising detecting antibodies against a BSX protein or an immunogenic fragment or epitope thereof in a biological sample from said subject, wherein a level of the antibodies that is enhanced compared to the level of the antibodies detectable in a normal or healthy subject indicates that the subject is not responding to said treatment or has not been rendered free of disease or infection.

In an alternative embodiment, the present invention provides a method for determining the response of a subject having tuberculosis or an infection by M. tuberculosis to treatment with a therapeutic compound for said tuberculosis or infection, said method comprising detecting antibodies against a BSX protein or an immunogenic fragment or epitope thereof in a biological sample from said subject, wherein a level of the antibodies that is lower than the level of the antibodies detectable in a subject suffering from tuberculosis or infection by M. tuberculosis indicates that the subject is responding to said treatment or has been rendered free of disease or infection.

The amount of an antibody against the BSX protein or fragment that is detected in a biological sample from a subject with tuberculosis may be compared to a reference sample, wherein the reference sample is derived from one or more healthy subjects who have not been previously infected with M. tuberculosis or not recently-infected with M. tuberculosis. Such negative control subjects will have a low circulating antibody titer making them suitable standards in antibody-based assay formats. For example, antibodies against a BSX protein or immunogenic fragment thereof are not detected in the reference sample and only in a patient sample, indicating that the patient from whom the sample was derived is suffering from tuberculosis or infection by M. tuberculosis or will develop an acute infection.

In one embodiment of the diagnostic/prognostic methods described herein, the biological sample is obtained previously from the subject. In accordance with such an embodiment, the prognostic or diagnostic method is performed ex vivo.

In yet another embodiment, the subject diagnostic/prognostic methods further comprise processing the sample from the subject to produce a derivative or extract that comprises the analyte (e.g., blood, serum or any immunoglobulin-containing sample).

Suitable samples include, for example, extracts from tissues comprising an immunoglobulin such as blood, bone, or body fluids such as serum, plasma, whole blood, an immunoglobulin-containing fraction of serum, an immunoglobulin-containing fraction of plasma, an immunoglobulin-containing fraction of blood.

### 3. Detection Systems

Preferred detection systems contemplated herein include any known assay for detecting proteins or antibodies in a biological sample isolated from a human subject, such as, for example, SDS/PAGE, isoelectric focussing, 2-dimensional gel electrophoresis comprising SDS/PAGE and isoelectric focussing, an immunoassay, a detection based system using an antibody or non-antibody ligand of the protein, such as, for example, a small molecule (e.g. a chemical compound, agonist, antagonist, allosteric modulator, competitive inhibitor, or non-competitive inhibitor, of the protein). In accordance with these embodiments, the antibody or small molecule may be used in any standard solid phase or solution phase assay format amenable to the detection of proteins. Optical or fluorescent detection, such as, for example, using mass spectrometry, MALDI-TOF, biosensor technology, evanescent fiber optics, or fluorescence resonance energy transfer, is clearly encompassed by the present invention. Assay systems suitable for use in high throughput screening of mass samples, particularly a high throughput spectroscopy resonance method (e.g. MALDI-TOF, electrospray MS or nano-electrospray MS), are particularly contemplated.

Immunoassay formats are particularly preferred, e.g., selected from the group consisting of, an immunoblot, a Western blot, a dot blot, an enzyme linked immunosorbent assay (ELISA), radioimmunoassay (RIA), enzyme immunoassay. Modified immunoassays utilizing fluorescence resonance energy transfer (FRET), isotope-coded affinity tags (ICAT), matrix-assisted laser desorption/ionization time of flight (MALDI-TOF), electrospray ionization (ESI), biosensor technology, evanescent fiber-optics technology or protein chip technology are also useful.

Preferably, the assay is a semi-quantitative assay or quantitative assay.

Standard solid phase ELISA formats are particularly useful in determining the concentration of a protein or antibody from a variety of patient samples.

In one form such as an assay involves immobilising a biological sample comprising anti-BSX antibodies, or alternatively BSX protein or an immunogenic fragment thereof, onto a solid matrix, such as, for example a polystyrene or polycarbonate microwell or dipstick, a membrane, or a glass support (e.g. a glass slide).

In the case of an antigen-based assay, an immobilised antibody that specifically binds a BSX protein is brought into direct contact with the biological sample, and forms a direct bond with any of its target protein present in said sample. For an antibody-based assay, an immobilised isolated or recombinant BSX protein or an immunogenic fragment or epitope thereof will be contacted with the biological sample. The added antibody or protein in solution is generally labelled with a detectable reporter molecule, such as for example, a fluorescent label (e.g. FITC or Texas Red) or an enzyme (e.g. horseradish peroxidase (HRP)), alkaline phosphatase (AP) or β-galactosidase. Alternatively, or in addition, a second labelled antibody can be used that binds to the first antibody or to the isolated/recombinant BSX antigen. Following washing to remove any unbound antibody or BSX antigen, the label may be detected either directly, in the case of a fluorescent label, or through the addition of a substrate, such as for example hydrogen peroxide, TMB, or toluidine, or 5-bromo-4-chloro-3-indol-beta-D-galaotopyranoside (x-gal).

Such ELISA based systems are particularly suitable for quantification of the amount of a protein or antibody in a sample, such as, for example, by calibrating the detection system against known amounts of a standard.

In another form, an ELISA consists of immobilizing an antibody that specifically binds a BSX protein on a solid matrix, such as, for example, a membrane, a polystyrene or polycarbonate microwell, a polystyrene or polycarbonate dipstick or a glass support. A patient sample is then brought into physical relation with said antibody, and the antigen in the sample is bound or ‘captured’. The bound protein can then be detected using a labelled antibody. For example if the protein is captured from a human sample, an anti-human antibody is used to detect the captured protein.

In one example, the present invention comprises:
(i) immobilizing an antibody that specifically binds an immunogenic BSX peptide of the invention to a solid matrix or support (e.g., a peptide comprising a sequence set forth in any one or more of SEQ ID NOs: 2-53);
(ii) contacting the bound antibody with a sample obtained from a subject, preferably an antibody-containing sample such as blood, serum or Ig-containing fraction thereof for a time and under conditions sufficient for the immobilized antibody to bind to a GS protein or fragment thereof in the sample thereby forming an antigen-antibody complex; and
(iii) detecting the antigen-antibody complex formed in a process comprising contacting said complex with an antibody that recognizes human Ig, wherein the presence of said human Ig indicates the presence of M. tuberculosis in the patient sample.

In accordance with this embodiment, specificity of the immobilized antibody ensures that only isolated or immunocomplexed BSX protein or fragments comprising the epitope that the antibody recognizes actually bind, whilst specificity of anti-human Ig ensures that only immunocomplexed BSX protein or fragment is detected. In this context, the term “immunocomplexed” shall be taken to mean that the BSX protein or fragments thereof in the patient sample are complexed with human Ig such as human IgA or human IgM or human IgG, etc. Accordingly, this embodiment is particularly useful for detecting the presence of M. tuberculosis or an infection by M. tuberculosis that has produced an immune response in a subject. By appropriately selecting the detection antibody, e.g., anti-human IgA or anti-human IgG or anti-human IgM, it is further possible to isotype the immune response of the subject. Such detection antibodies against human IgA, IgM and IgG are publicly available to the art.

Alternatively or in addition to the preceding embodiments, a third labelled antibody can be used that binds the second (detecting) antibody.

It will be apparent to the skilled person that the assay formats described herein are amenable to high throughput formats, such as, for example automation of screening processes, or a microarray format as described in Mendoza et al, Biotechniques 27(4): 778-788, 1999. Furthermore, variations of the above described assay will be apparent to those skilled in the art, such as, for example, a competitive ELISA.

Alternatively, the presence of anti-BSX antibodies, or alternatively a BSX protein or an immunogenic fragment thereof, is detected using a radioimmunoassay (RIA). The basic principle of the assay is the use of a radiolabelled antibody or antigen to detect antibody antigen interactions. For example, an antibody that specifically binds to a BSX protein can be bound to a solid support and a biological sample brought into direct contact with said antibody. To detect the bound antigen, an isolated and/or recombinant form of the antigen is radiolabelled is brought into contact with the same antibody. Following washing the amount of bound radioactivity is detected. As any antigen in the biological sample inhibits binding of the radiolabelled antigen the amount of radioactivity detected is inversely proportional to the amount of antigen in the sample. Such an assay may be quantitated by using a standard curve using increasing known concentrations of the isolated antigen.

As will be apparent to the skilled artisan, such an assay may be modified to use any reporter molecule, such as, for example, an enzyme or a fluorescent molecule, in place of a radioactive label.

Western blotting is also useful for detecting a BSX protein or an immunogenic fragment thereof. In such an assay, protein from a biological sample is separated using sodium dodecyl sulphate (SDS) polyacrylamide gel electrophoresis (SDS-PAGE) using techniques well known in the art and described in, for example, Scopes (In: Protein Purification: Principles and Practice, Third Edition, Springer Verlag, 1994). Separated proteins are then transferred to a solid support, such as, for example, a membrane or more specifically PVDF membrane, using methods well known in the art, for example, electrotransfer. This membrane may then be blocked and probed with a labelled antibody or ligand that specifically binds a BSX protein. Alternatively, a labelled secondary, or even tertiary, antibody or ligand can be used to detect the binding of a specific primary antibody.

High-throughput methods for detecting the presence or absence of anti-BSX antibodies, or alternatively BSX protein or an immunogenic fragment thereof are particularly preferred.

In one embodiment, MALDI-TOF is used for the rapid identification of a protein that has been separated by either one- or two-dimensional gel electrophoresis. Accordingly, there is no need to detect the proteins of interest using an antibody or ligand that specifically binds to the protein of interest. Rather, proteins from a biological sample are separated using gel electrophoresis using methods well known in the art and those proteins at approximately the correct molecular weight and/or isoelectric point are analysed using MALDI-TOF to determine the presence or absence of a protein of interest.

Alternatively, MALDI or ESI or a combination of approaches is used to determine the concentration of a particular protein in a biological sample, such as, for example sputum. Such proteins are preferably well characterised previously with regard to parameters such as molecular weight and isoelectric point.

Biosensor devices generally employ an electrode surface in combination with current or impedance measuring elements to be integrated into a device in combination with the assay substrate (such as that described in U.S. Pat. No. 5,567,301). An antibody or ligand that specifically binds to a protein of interest is preferably incorporated onto the surface of a biosensor device and a biological sample isolated from a patient (for example sputum that has been solubilised using the methods described herein) contacted to said device. A change in the detected current or impedance by the biosensor device indicates protein binding to said antibody or ligand. Some forms of biosensors known in the art also rely on surface plasmon resonance to detect protein interactions, whereby a change in the surface plasmon resonance surface of reflection is indicative of a protein binding to a ligand or antibody (U.S. Pat. Nos. 5,485,277 and 5,492,840).

Biosensors are of particular use in high throughput analysis due to the ease of adapting such systems to micro- or nano-scales. Furthermore, such systems are conveniently adapted to incorporate several detection reagents, allowing for multiplexing of diagnostic reagents in a single biosensor unit. This permits the simultaneous detection of several epitopes in a small amount of body fluids.

Evanescent biosensors are also preferred as they do not require the pretreatment of a biological sample prior to detection of a protein of interest. An evanescent biosensor generally relies upon light of a predetermined wavelength interacting with a fluorescent molecule, such as for example, a fluorescent antibody attached near the probe's surface, to emit fluorescence at a different wavelength upon binding of the diagnostic protein to the antibody or ligand.

To produce protein chips, the proteins, peptides, polypeptides, antibodies or ligands that are able to bind specific antibodies or proteins of interest are bound to a solid support such as for example glass, polycarbonate, polytetrafluoroethylene, polystyrene, silicon oxide, metal or silicon nitride. This immobilization is either direct (e.g. by covalent linkage, such as, for example, Schiff's base formation, disulfide linkage, or amide or urea bond formation) or indirect. Methods of generating a protein chip are known in the art and are described in for example U.S. Patent Application No. 20020136821, 20020192654, 20020102617 and U.S. Pat. No. 6,391,625. In order to bind a protein to a solid support it is often necessary to treat the solid support so as to create chemically reactive groups on the surface, such as, for example, with an aldehyde-containing silane reagent. Alternatively, an antibody or ligand may be captured on a microfabricated polyacrylamide gel pad and accelerated into the gel using microelectrophoresis as described in, Arenkov et al. Anal. Biochem. 278:123-131, 2000.

A protein chip is preferably generated such that several proteins, ligands or antibodies are arrayed on said chip. This format permits the simultaneous screening for the presence of several proteins in a sample.

Alternatively, a protein chip may comprise only one protein, ligand or antibody, and be used to screen one or more patient samples for the presence of one polypeptide of interest. Such a chip may also be used to simultaneously screen an array of patient samples for a polypeptide of interest.

Preferably, a sample to be analysed using a protein chip is attached to a reporter molecule, such as, for example, a fluorescent molecule, a radioactive molecule, an enzyme, or an antibody that is detectable using methods well known in the art. Accordingly, by contacting a protein chip with a labelled sample and subsequent washing to remove any unbound proteins the presence of a bound protein is detected using methods well known in the art, such as, for example using a DNA microarray reader.

Alternatively, biomolecular interaction analysis-mass spectrometry (BIA-MS) is used to rapidly detect and characterise a protein present in complex biological samples at the low- to sub-femptamole (fmol) level (Nelson et al. Electrophoresis 21: 1155-1163, 2000). One technique useful in the analysis of a protein chip is surface enhanced laser desorption/ionization-time of flight-mass spectrometry (SELDI-TOF-MS) technology to characterise a protein bound to the protein chip. Alternatively, the protein chip is analysed using ESI as described in U.S. Patent Application 20020139751.

As will be apparent to the skilled artisan, protein chips are particularly amenable to multiplexing of detection reagents. Accordingly, several antibodies or ligands each able to specifically bind a different peptide or protein may be bound to different regions of said protein chip. Analysis of a biological sample using said chip then permits the detecting of multiple proteins of interest, or multiple B cell epitopes of the BSX protein. Multiplexing of diagnostic and prognostic markers is particularly contemplated in the present invention.

In a further embodiment, the samples are analysed using ICAT, essentially as described in US Patent Application No. 20020076739. This system relies upon the labelling of a protein sample from one source (i.e. a healthy individual) with a reagent and the labelling of a protein sample from another source (i.e. a tuberculosis patient) with a second reagent that is chemically identical to the first reagent, but differs in mass due to isotope composition. It is preferable that the first and second reagents also comprise a biotin molecule. Equal concentrations of the two samples are then mixed, and peptides recovered by avidin affinity chromatography. Samples are then analysed using mass spectrometry. Any difference in peak heights between the heavy and light peptide ions directly correlates with a difference in protein abundance in a biological sample. The identity of such proteins may then be determined using a method well known in the art, such as, for example MALDI-TOF, or ESI.

In a particularly preferred embodiment, a biological sample comprising anti-BSX antibodies, or alternatively BSX protein or an immunogenic fragment thereof, is subjected to 2-dimensional gel electrophoresis. In accordance with this embodiment, it is preferable to remove certain particulate matter from the sample prior to electrophoresis, such as, for example, by centrifugation, filtering, or a combination of centrifugation and filtering. Proteins in the biological sample are then separated. For example, the proteins may be separated according to their charge using isoelectric focussing and/or according to their molecular weight. Two-dimensional separations allow various isoforms of proteins to be identified, as proteins with similar molecular weight are also separated by their charge. Using mass spectrometry, it is possible to determine whether or not a protein of interest is present in a patient sample.

As will be apparent to those skilled in the art a diagnostic or prognostic assay described herein may be a multiplexed assay. As used herein the term “multiplex”, shall be understood not only to mean the detection of two or more diagnostic or prognostic markers in a single sample simultaneously, but also to encompass consecutive detection of two or more diagnostic or prognostic markers in a single sample, simultaneous detection of two or more diagnostic or prognostic markers in distinct but matched samples, and consecutive detection of two or more diagnostic or prognostic markers in distinct but matched samples. As used herein the term “matched samples” shall be understood to mean two or more samples derived from the same initial biological sample, or two or more biological samples isolated at the same point in time.

Accordingly, a multiplexed assay may comprise an assay that detects several anti-BSX antibodies and/or BSX epitopes in the same reaction and simultaneously, or alternatively, it may detect other one or more antigens/antibodies in addition to one or more anti-BSX antibodies and/or BSX epitopes.

The present invention clearly contemplates multiplexed assays for detecting BSX antibodies and epitopes in addition to detecting CD4+ T-helper cells via one or more receptors on the cell surface and/or one or more HIV-1 and/or HUV-2 antigens. Such assays are particularly useful for simultaneously obtaining information on co-infection with M. tuberculosis and HIV-1 and/or HIV-2, and/or for determining whether or not a subject with M. tuberculosis is immune-compromised. Clearly, such multiplexed assay formats are useful for monitoring the health of an HIV+/TB+ individual.

As will be apparent to the skilled artisan, if such an assay is antibody or ligand based, both of these antibodies must function under the same conditions.

### 4. Biological Samples and Reference Samples

Preferably the biological sample in which a BSX protein or anti-BSX antibody is detected is a sample selected from the group consisting of lung, lymphoid tissue associated with the lung, paranasal sinuses, bronchi, a bronchiole, alveolus, ciliated mucosal epithelia of the respiratory tract, mucosal epithelia of the respiratory tract, broncheoalveolar lavage fluid (BAL), alveolar lining fluid, sputum, mucus, saliva, blood, serum, plasma, urine, peritoneal fluid, pericardial fluid, pleural fluid, squamous epithelial cells of the respiratory tract, a mast cell, a goblet cell, a pneumocyte (type 1 or type 2), an intra epithelial dendritic cell, a PBMC, a neutrophil, a monocyte, or any immunoglobulin-containing fraction of any one or more of said tissues, fluids or cells.

In one embodiment a biological sample is obtained previously from a patient.

In one embodiment a biological sample is obtained from a subject by a method selected from the group consisting of surgery or other excision method, aspiration of a body fluid such as hypertonic saline or propylene glycol, broncheoalveolar lavage, bronchoscopy, saliva collection with a glass tube, salivette (Sarstedt AG, Sevelen, Switzerland), Ora-sure (Epitope Technologies Pty Ltd, Melbourne, Victoria, Australia), omni-sal (Saliva Diagnostic Systems, Brooklyn, N.Y., USA) and blood collection using any method well known in the art, such as, for example using a syringe.

It is particularly preferred that a biological sample is sputum, isolated from lung of a patient using, for example the method described in Gershman, N. H. et al, J Allergy Clin Immunol, 10(4): 322-328, 1999.

In another preferred embodiment a biological sample is plasma that has been isolated from blood collected from a patient using a method well known in the art.

In one embodiment, a biological sample is treated to lyse a cell in said sample. Such methods include the use of detergents, enzymes, repeatedly freezing and thawing said cells, sonication or vortexing said cells in the presence of glass beads, amongst others.

In another embodiment, a biological sample is treated to denature a protein present in said sample. Methods of denaturing a protein include heating a sample, treating a sample with 2-mercaptoethanol, dithiotreitol (DTT), detergent or other compound such as, for example, guanidinium or urea. For example, the use of DTT is preferred for liquefying sputum.

In yet another embodiment, a biological sample is treated to concentrate a protein is said sample. Methods of concentrating proteins include precipitation, freeze drying, use of funnel tube gels (TerBush and Novick, Journal of Biomolecular Techniques, 10(3); 1999), ultrafiltration or dialysis.

As will be apparent, the diagnostic and prognostic methods provided by the present invention require a degree of quantification to determine either, the amount of a protein that is diagnostic or prognostic of an infection or disease. Such quantification can be determined by the inclusion of appropriate reference samples in the assays described herein, wherein said reference samples are derived from healthy or normal individuals.

In one embodiment, the reference sample comprises for example cells, fluids or tissues from a healthy subject who has not been previously or recently infected and is not suffering from an infection or disease. Conveniently, such reference samples are from fluids or tissues that do not require surgical resection or intervention to obtain them. Accordingly, bodily fluids and derivatives thereof are preferred. Highly preferred reference samples comprise sputum, mucus, saliva, blood, serum, plasma, urine, BAL fluid, peritoneal fluid, pericardial fluid, pleural fluid, a PBMC, a neutrophil, a monocyte, or any immunoglobulin-containing fraction of any one or more of said tissues, fluids or cells.

A reference sample and a test (or patient) sample are both processed, analysed or assayed and data obtained for a reference sample and a test sample are compared. In one embodiment, a reference sample and a test sample are processed, analysed or assayed at the same time. In another embodiment, a reference sample and a test sample are processed, analysed or assayed at a different time.

In an alternate embodiment, a reference sample is not included in an assay. Instead, a reference sample may be derived from an established data set that has been previously generated. Accordingly, in one embodiment, a reference sample comprises data from a sample population study of healthy individuals, such as, for example, statistically significant data for the healthy range of the integer being tested. Data derived from processing, analysing or assaying a test sample is then compared to data obtained for the sample population.

Data obtained from a sufficiently large number of reference samples so as to be representative of a population allows the generation of a data set for determining the average level of a particular parameter. Accordingly, the amount of a protein that is diagnostic or prognostic of an infection or disease can be determined for any population of individuals, and for any sample derived from said individual, for subsequent comparison to levels of the expression product determined for a sample being assayed. Where such normalized data sets are relied upon, internal controls are preferably included in each assay conducted to control for variation.

### Diagnostic Assay Kits

The present invention provides a kit for detecting M. tuberculosis infection in a biological sample. In one embodiment, the kit comprises:
(i) one or more isolated antibodies that bind to a BSX protein or an immunogenic fragment or epitope thereof; and
(ii) means for detecting the formation of an antigen-antibody complex.

In an alternative embodiment, the kit comprises:
(i) an isolated or recombinant BSX protein or an immunogenic fragment or epitope thereof; and
(ii) means for detecting the formation of an antigen-antibody complex.

The antibodies, immunogenic BSX peptide, and detection means of the subject kit are preferably selected from the antibodies and immunogenic BSX peptides described herein above and those embodiments shall be taken to be incorporated by reference herein from the description. The selection of compatible kit components for any assay format will be readily apparent to the skilled artisan from the description.

In a particularly preferred embodiment, the subject kit comprises:
(i) an antibody that binds to an isolated or recombinant or synthetic peptide comprising an amino acid sequence selected from the group consisting of SEQ ID NOs: 24, 25 and 45; and
(ii) anti-human Ig.

Preferably, the kit further comprises an amount of one or more other peptides each comprising an amino acid sequence selected from the group consisting of SEQ ID NOs: 2-23, 26-44 and 46-55, or a fusion between any two or more of said peptides.

Optionally, the kit further comprises means for the detection of the binding of an antibody, fragment thereof or a ligand to a BSX protein. Such means include a reporter molecule such as, for example, an enzyme (such as horseradish peroxidase or alkaline phosphatase), a substrate, a cofactor, an inhibitor, a dye, a radionucleotide, a luminescent group, a fluorescent group, biotin or a colloidal particle, such as colloidal gold or selenium. Preferably such a reporter molecule is directly linked to the antibody or ligand.

In yet another embodiment, a kit may additionally comprise a reference sample. Such a reference sample may for example, be a protein sample derived from a biological sample isolated from one or more tuberculosis subjects. Alternatively, a reference sample may comprise a biological sample isolated from one or more normal healthy individuals. Such a reference sample is optionally included in a kit for a diagnostic or prognostic assay.

In another embodiment, a reference sample comprises a peptide that is detected by an antibody or a ligand. Preferably, the peptide is of known concentration. Such a peptide is of particular use as a standard. Accordingly various known concentrations of such a peptide may be detected using a prognostic or diagnostic assay described herein.

In yet another embodiment, a kit optionally comprises means for sample preparations, such as, for example, a means for cell lysis. Preferably such means are means of solubilizing sputum, such as, for example, a detergent (e.g. tributyl phosphine, C7BZO, dextran sulfate, or polyoxyethylenesorbitan monolaurate.

In yet another embodiment, a kit comprises means for protein isolation (Scopes (In: Protein Purification Principles and Practice, Third Edition, Springer Verlag, 1994).

### Prophylactic and Therapeutic Method

The BSX protein or immunogenic fragment or epitope thereof can induce the specific production of a high titer antibody when administered to an animal subject.

Accordingly, the invention provides a method of eliciting the production of antibody against M. tuberculosis comprising administering an isolated BSX protein or an immunogenic fragment or epitope thereof to said subject for a time and under conditions sufficient to elicit the production of antibodies, such as, for example, neutralizing antibodies against M. tuberculosis.

Preferably, the neutralizing antibodies are high titer neutralizing antibodies.

The effective amount of BSX protein or epitope to produce antibodies varies upon the nature of the immunogenic B cell epitope, the route of administration, the animal used for immunization, and the nature of the antibody sought. All such variables are empirically determined by art-recognized means.

In a preferred embodiment, the invention provides a method of inducing immunity against M. tuberculosis in a subject comprising administering to said subject an isolated or recombinant BSX protein or immunogenic fragment or epitope thereof for a time and under conditions sufficient to elicit a humoral immune response against said an isolated or recombinant BSX protein or immunogenic fragment or epitope.

The immunizing antigen may be administered in the form of any convenient formulation as described herein.

By “humoral immune response” means that a secondary immune response is generated against the immunizing antigen sufficient to prevent infection by M. tuberculosis.

Preferably, the humoral immunity generated includes eliciting in the subject a sustained level of antibodies against a B cell epitope in the immunizing antigen. By a “sustained level of antibodies” is meant a sufficient level of circulating antibodies against the B cell epitope to prevent infection by M. tuberculosis.

Preferably, antibodies levels are sustained for at least about six months or 9 months or 12 months or 2 years.

In an alternative embodiment, the present invention provides a method of enhancing the immune system of a subject comprising administering an immunologically active BSX protein or an epitope thereof or a vaccine composition comprising said BSX protein or epitope for a time and under conditions sufficient to confer or enhance resistance against M. tuberculosis in said subject.

By “confer or enhance resistance” is meant that a M. tuberculosis-specific immune response occurs in said subject, said response being selected from the group consisting of:
(i) an antibody against a BSX protein of M. tuberculosis or an epitope of said protein is produced in said subject;
(ii) neutralizing antibodies against M. tuberculosis are produced in said subject;
(iii) a cytotoxic T lymphocyte (CTL) and/or a CTL precursor that is specific for a BSX protein of M. tuberculosis is activated in the subject; and
(iv) the subject has enhanced immunity to a subsequent M. tuberculosis infection or reactivation of a latent M. tuberculosis infection.

The invention will be understood to encompass a method of providing or enhancing immunity against M. tuberculosis in an uninfected human subject comprising administering to said subject an immunologically active BSX protein or an epitope thereof or a vaccine composition comprising said BSX protein or epitope for a time and under conditions sufficient to provide immunological memory against a future infection by M. tuberculosis.

The present invention provides a method of treatment of tuberculosis in a subject comprising performing a diagnostic method or prognostic method as described herein.

In one embodiment, the present invention provides a method of prophylaxis comprising:
(i) detecting the presence of M. tuberculosis infection in a biological sample from a subject; and
(ii) administering a therapeutically effective amount of a pharmaceutical composition to reduce the number of pathogenic bacilli in the lung, blood or lymph system of the subject.

Preferably, the BSX protein or epitope or vaccine is administered to a subject harboring a latent or active M. tuberculosis infection.

Without being bound by any theory or mode of action, the therapeutic method enhances the ability of a T cell to recognize and lyse a cell harboring M. tuberculosis, or that the ability of a T cell to recognize a T cell epitope of an antigen of M. tuberculosis is enhanced, either transiently or in a sustained manner. Similarly, reactivation of a T cell population may occur following activation of a latent M. tuberculosis infection, or following re-infection with M. tuberculosis, or following immunization of a previously-infected subject with a BSX protein or epitope or vaccine composition of the invention. Standard methods can be used to determine whether or not CTL activation has occurred in the subject, such as, for example, using cytotoxicity assays, ELISPOT, or determining IFN-γ production in PBMC of the subject.

Preferably, the peptide or derivative or variant or vaccine composition is administered for a time and under conditions sufficient to elicit or enhance the expansion of CD8⁺ T cells. Still more preferably, the peptide or derivative or variant or vaccine composition is administered for a time and under conditions sufficient for M. tuberculosis-specific cell mediated immunity (CMI) to be enhanced in the subject.

By “M. tuberculosis-specific CMI” is meant that the activated and clonally expanded CTLs are MHC-restricted and specific for a CTL epitope of the invention. CTLs are classified based on antigen specificity and MHC restriction, (i.e., non-specific CTLs and antigen-specific, MHC-restricted CTLs). Non-specific CTLs are composed of various cell types, including NK cells and antibody-dependent cytotoxicity, and can function very early in the immune response to decrease pathogen load, while antigen-specific responses are still being established. In contrast, MHC-restricted CTLs achieve optimal activity later than non-specific CTL, generally before antibody production. Antigen-specific CTLs inhibit or reduce the spread of M. tuberculosis and preferably terminate infection.

CTL activation, clonal expansion, or CMI can be induced systemically or compartmentally localized. In the case of compartmentally localized effects, it is preferred to utilize a vaccine composition suitably formulated for administration to that compartment. On the other hand, there are no such stringent requirements for inducing CTL activation, expansion or CMI systemically in the subject.

The effective amount of BSX protein or epitope thereof to be administered, either solus or in a vaccine composition to elicit CTL activation, clonal expansion or CMI varies upon the nature of the immunogenic epitope, the route of administration, the weight, age, sex, or general health of the subject immunized, and the nature of the CTL response sought. All such variables are empirically determined by art-recognized means.

The BSX protein or an epitope thereof, optionally formulated with any suitable or desired carrier, adjuvant, BRM, or pharmaceutically acceptable excipient, is conveniently administered in the form of an injectable composition. Injection may be intranasal, intramuscular, sub-cutaneous, intravenous, intradermal, intraperitoneal, or by other known route. For intravenous injection, it is desirable to include one or more fluid and nutrient replenishers.

The optimum dose to be administered and the preferred route for administration are established using animal models, such as, for example, by injecting a mouse, rat, rabbit, guinea pig, dog, horse, cow, goat or pig, with a formulation comprising the peptide, and then monitoring the CTL immune response to the epitope using any conventional assay.

Adoptive transfer techniques may also be used to confer or enhance resistance against M. tuberculosis infection or to prevent or reduce the severity of a reactivated latent infection. Accordingly, in a related embodiment, there is provided a method of enhancing or conferring immunity against M. tuberculosis in an uninfected human subject comprising contacting ex vivo a T cell obtained from a human subject with an immunologically active BSX protein or an epitope thereof or a vaccine composition comprising said protein or epitope for a time and under conditions sufficient to confer M. tuberculosis activity on said T cells.

In a preferred embodiment, the invention provides a method of enhancing the M. tuberculosis-specific cell mediated immunity of a human subject, said method comprising:
(i) contacting ex vivo a T cell obtained from a human subject with an immunologically active BSX protein or a CTL epitope thereof or a vaccine composition comprising said protein or epitope for a time and under conditions sufficient to confer M. tuberculosis activity on said T cells; and
(ii) introducing the activated T cells autologously to the subject or allogeneically to another human subject.

The T cell may be a CTL or CTL precursor cell.

The human subject from whom the T cell is obtained may be the same subject or a different subject to the subject being treated. The subject being treated can be any human subject carrying a latent or active M. tuberculosis infection or at risk of M. tuberculosis infection or reactivation of M. tuberculosis infection or a person who is otherwise in need of obtaining vaccination against M. tuberculosis or desirous of obtaining vaccination against M. tuberculosis.

Such adoptive transfer is preferably carried out and M. tuberculosis reactivity assayed essentially as described by Einsele et al., Blood 99, 3916-3922, 2002, which procedures are incorporated herein by reference.

By “M. tuberculosis activity” is meant that the T cell is rendered capable of being activated as defined herein above (i.e. the T cell will recognize and lyse a cell harboring M. tuberculosis or able to recognize a T cell epitope of an antigen of M. tuberculosis, either transiently or in a sustained manner). Accordingly, it is particularly preferred for the T cell to be a CTL precursor which by the process of the invention is rendered able to recognize and lyse a cell harboring M. tuberculosis or able to recognize a T cell epitope of an antigen of M. tuberculosis, either transiently or in a sustained manner.

For such an ex vivo application, the T cell is preferably contained in a biological sample obtained from a human subject, such as, for example, a biopsy specimen comprising a primary or central lymphoid organ (e.g. bone marrow or thymus) or a secondary or peripheral lymphoid organ (e.g. blood, PBMC or a buffy coat fraction derived there from).

Preferably, the T cell or specimen comprising the T cell was obtained previously from a human subject, such as, for example, by a consulting physician who has referred the specimen to a pathology laboratory for analysis.

Preferably, the subject method further comprises obtaining a sample comprising the T cell of the subject, and more preferably, obtaining said sample from said subject.

### Formulations

The present invention clearly contemplates the use of the BSX protein or an immunogenic fragment or epitope thereof in the preparation of a therapeutic or prophylactic subunit vaccine against M. tuberculosis infection in a human or other animal subject.

Accordingly, the invention provides a pharmaceutical composition or vaccine comprising a BSX protein or an immunogenic fragment or epitope thereof in combination with a pharmaceutically acceptable diluent.

The BSX protein or immunogenic fragment or epitope thereof is conveniently formulated in a pharmaceutically acceptable excipient or diluent, such as, for example, an aqueous solvent, non-aqueous solvent, non-toxic excipient, such as a salt, preservative, buffer and the like. Examples of non-aqueous solvents are propylene glycol, polyethylene glycol, vegetable oil and injectable organic esters such as ethyloleate. Aqueous solvents include water, alcoholic/aqueous solutions, saline solutions, parenteral vehicles such as sodium chloride, Ringer's dextrose, etc. Preservatives include antimicrobial, anti-oxidants, chelating agents and inert gases. The pH and exact concentration of the various components the pharmaceutical composition are adjusted according to routine skills in the art.

In certain situations, it may also be desirable to formulate the BSX protein or immunogenic fragment or epitope thereof with an adjuvant to enhance the immune response to the B cell epitope. Again, this is strictly not essential. Such adjuvants include all acceptable immunostimulatory compounds such as, for example, a cytokine, toxin, or synthetic composition. Exemplary adjuvants include IL-1, IL-2, BCG, aluminium hydroxide, N-acetyl-muramyl-L-threonyl-D-isoglutamine (thur-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP) 1983A, referred to as MTP-PE), lipid A, MPL and RMI, which contains three components extracted from bacteria, monophosphoryl lipid A, trehalose dimycolate and cell wall skeleton (MPL+TDM+CWS) in a 2% squalene/Tween 80 emulsion.

Particularly preferred adjuvants for use in a vaccine against M. tuberculosis are described for example by Elhay and Andersen Immunol. Cell Biol. 75, 595-603, 1997; or Lindblad et al., Infect. Immun. 65, 1997.

It may be desirable to co-administer biologic response modifiers (BRM) with the BSX protein or immunogenic fragment or epitope thereof, to down regulate suppressor T cell activity. Exemplary BRM's include, but are not limited to, Cimetidine (CIM; 1200 mg/d) (Smith/Kline, Pa., USA); Indomethacin (IND; 150 mg/d) (Lederle, N.J., USA); or low-dose Cyclophosphamide (CYP; 75, 150 or 300 mg/m.sup.2) (Johnson/Mead, N.J., USA).

Preferred vehicles for administration of the BSX protein or immunogenic fragment or epitope thereof include liposomes. Liposomes are microscopic vesicles that consist of one or more lipid bilayers surrounding aqueous compartments. (Bakker-Woudenberg et al., Eur. J. Clin. Microbiol. Infect. Dis. 12(Suppl. 1), S61 (1993); and Kim, Drugs 46, 618 (1993)). Liposomes are similar in composition to cellular membranes and as a result, liposomes generally are administered safely and are biodegradable.

Techniques for preparation of liposomes and the formulation (e.g., encapsulation) of various molecules, including peptides and oligonucleotides, with liposomes are well known to the skilled artisan.

Depending on the method of preparation, liposomes may be unilamellar or multilamellar, and can vary in size with diameters ranging from 0.02 μm to greater than 10 μm. A variety of agents are encapsulated in liposomes. Hydrophobic agents partition in the bilayers and hydrophilic agents partition within the inner aqueous space(s) (Machy et al., LIPOSOMES IN CELL BIOLOGY AND PHARMACOLOGY (John Libbey 1987), and Ostro et al., American J. Hosp. Pharm. 46, 1576 (1989)).

Liposomes can also adsorb to virtually any type of cell and then release the encapsulated agent. Alternatively, the liposome fuses with the target cell, whereby the contents of the liposome empty into the target cell. Alternatively, an absorbed liposome may be endocytosed by cells that are phagocytic. Endocytosis is followed by intralysosomal degradation of liposomal lipids and release of the encapsulated agents (Scherphof et al., Ann. N.Y. Acad. Sci. 446, 368 (1985)). In the present context, the BSX protein or immunogenic fragment or epitope thereof may be localized on the surface of the liposome, to facilitate antigen presentation without disruption of the liposome or endocytosis. Irrespective of the mechanism or delivery, however, the result is the intracellular disposition of the associated BSX protein or immunogenic fragment or epitope thereof.

Liposomal vectors may be anionic or cationic. Anionic liposomal vectors include pH sensitive liposomes which disrupt or fuse with the endosomal membrane following endocytosis and endosome acidification. Cationic liposomes are preferred for mediating mammalian cell transfection in vitro, or general delivery of nucleic acids, but are used for delivery of other therapeutics, such as peptides or lipopeptides.

Cationic liposome preparations are made by conventional methodologies (Feigner et al, Proc. Nat'l Acad. Sci. USA 84, 7413 (1987); Schreier, Liposome Res. 2, 145 (1992)). Commercial preparations, such as Lipofectin (Life Technologies, Inc., Gaithersburg, Md. USA), are readily available. The amount of liposomes to be administered are optimized based on a dose response curve. Feigner et al., supra.

Other suitable liposomes that are used in the methods of the invention include multilamellar vesicles (MLV), oligolamellar vesicles (OLV), unilamellar vesicles (UV), small unilamellar vesicles (SUV), medium-sized unilamellar vesicles (MUV), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV). The skilled artisan will recognize that the techniques for preparing these liposomes are well known in the art. (See COLLOIDAL DRUG DELIVERY SYSTEMS, vol. 66, J. Kreuter, ed., Marcel Dekker, Inc. 1994).

Other forms of delivery particle, for example, microspheres and the like, also are contemplated for delivery of the BSX protein or immunogenic fragment or epitope thereof.

Guidance in preparing suitable formulations and pharmaceutically effective vehicles, are found, for example, in REMINGTON'S PHARMACEUTICAL SCIENCES, chapters 83-92, pages 1519-1714 (Mack Publishing Company 1990) (Remington's), which are hereby incorporated by reference.

Alternatively, the peptide or derivative or variant is formulated as a cellular vaccine via the administration of an autologous or allogeneic antigen presenting cell (APC) or a dendritic cell that has been treated in vitro so as to present the peptide on its surface.

Nucleic acid-based vaccines that comprise nucleic acid, such as, for example, DNA or RNA, encoding the immunologically active BSX protein or epitope(s) and cloned into a suitable vector (e.g. vaccinia, canary pox, adenovirus, or other eukaryotic virus vector) are also contemplated. Preferably, DNA encoding a BSX protein is formulated into a DNA vaccine, such as, for example, in combination with the existing Calmette-Guerin (BCG) or an immune adjuvant such as vaccinia virus, Freund's adjuvant or another immune stimulant.

The present invention is further described with reference to the following non-limiting examples.

### Example 1

### Preparation of Sera for Proteomic Analysis

HIV⁺ and M. tuberculosis culture positive sera, and HIV⁺ M. tuberculosis culture negative sera, were independently thawed and processed. CHAPS was added to a final concentration of 0.5% (w/v). Samples were then mixed with 9 parts cold (−20° C.) acetone and precipitated for about 1 h at −20° C. The precipitate was pelleted at 5000 g for 20 min at 4° C., and resuspended by vortexing in 10 parts of 7 M urea, 2 M thiourea, CHAPS 2%, 5 mM Tris. The suspension was reduced with 5 mM tributylphosphine for 1 h at room temperature, then alkylated with 15 mM iodoacetamide for 1 h (iodoacetamide prepared freshly as a 300 mM solution).

### Example 2

### Analytical Methods

### Multi Compartmental Electrophoresis (MCE)

MCE membranes were prepared at pH 3, 5.5, 6.3 and 10.5 by combining 2.5 μl TEMED 5 μl and 40% APS to immobiline solution and soaking microfibre glass filter papers in this solution and leaving to dry at 50° C. for one hour. Once dry, the membranes were washed in 7M Urea 4 times for 30 minutes.

The reduced and alkylated samples were loaded into the central chamber of the MCE. The chambers adjacent to the central one were loaded with 7 M urea, 2 M thiourea and CHAPS 2%. The end acidic chamber was loaded with 7 M urea, 2 M thiourea and orthophosphoric acid (final conc. 0.28% v/v). The pH of the acidic chamber solution was about 2.5 and was adjusted with orthophosphoric acid so that it was about 0.5 pH unit lower than the pH of the adjacent chamber. The end alkaline chamber was loaded with 7 M urea, 2 M thiourea and approximately 7 mM sodium hydroxide (diluted from a 10 M stock). The pH of the alkaline chamber solution was adjusted to about 11, about 0.5 pH unit higher than the pH of the adjacent chamber. Each chamber was stirred vigorously during the experiment.

Samples were focused in the MCE for 40-48 h with a voltage range between 600-800 volts and a constant wattage of one. The sample was removed from unit and pipetted into 1 ml aliquots and stored at −80° C. Each large MCE run produced 3 fractions: acid (pI 3-5.5), albumin (5.5-6.3) and alkaline (6.3-10.5).

### First Dimension Electrophoresis:

Depending on experimental aims, samples were focused either with 7 cm or 11 cm IPG strips. The 7 cm strips were focused for between 45,000-50,000 Vh; 11 cm strips were focused for about 110,000 Vh.

### Second Dimension Electrophoresis:

All strips were electrophoresed using Invitrogen NuPage Bis-Tris 4-12%, 1.5 mm, 2D mini gels or alternatively, Proteome Systems 11 cm Gel Chips. In the case of 11 cm strips run in the Invitrogen NuPage Bis-Tris gels, the IPG strips were cut in half. Each half was loaded into separate gels. IPG strips were embedded in 0.5% agarose prepared in 1×MOPS gel running buffer, as described by Invitrogen. The gel running procedure was 30 min at 5 mA/gel, 30 min at 10 mA/gel and 30 mA/gel until the tracking dye was approximately 3 mm from base of gel. Proteome Systems Gel Chips were run in 50 mM Tris, 50 mM Tricine, 1% SDS at 50 mA per gel for 1.1 to 1.3 hrs. Gels were stained with G-250 colloidal coomassie blue or SyproRuby or silver. Gels stained with SyproRuby were first fixed in 2 changes of 7% acetic acid and 10% methanol, prior to staining, and were destained overnight in the same reagent.

### Mass Spectrometry:

Two-dimensional (2D) gels were imaged with the AlphaInnotech gel imager and/or scanned to file using a HP Scanjet 5200C. Protein spots from 2D gels were excised and placed in 150 μl 50% v/v acetonitrile (MeCN), sealed with tape and incubated for 1 hr at 30° C. to remove the coomassie stain. The MeCN was removed and the gel pieces dried in the oven for another 20 minutes. 5 μg/ml trypsin in 30 μl of 50 mM NH₄HCO₃ buffer was added to the gel pieces and digested overnight at 30° C. After overnight trypsin digestion, the peptides were extracted and automatically loaded to target plate using the MAP II/8. Following this step, leftover protein extractions were stored at −20° C. for possible further analysis. Note that the final MAP II/8 step could be omitted if material was intended for LC MS-MS analysis instead of MALDI or MALDI PSD analysis. Thus, depending on the aims of the experiment, in any combination the following MS techniques could be used either to i) identify or ii) verify a putative identification: MALDI-MS-TOF, MALDI-MS-TOF-PSD, and LC MS-MS, including nano-spray LC-MS-MS. For MALDI analysis either an Shimadzu Kratos Axima-CFR or a Bruker Biflex III instrument was used.

### Bioinformatic Analysis:

Following automated collection of mass spectra peaks, data were processed as follows. All spectra were firstly checked for correct calibration of peptide masses. Spectra were then processed to remove background noise including masses corresponding to trypsin peaks and matrix. The data were then searched against publicly-available SwissProt and TrEMBL databases using Proteome Systems search engine IonIQ v69. PSD data was searched against the same databases using the in-house search engine FragmentastIQ. LC MS-MS data was also searched against the databases using the SEQUEST search engine software.

### Example 3

### Identification of a Diagnostic Marker of M. Tuberculosis Infection

A protein having an isoelectric point of about 5.23 and a molecular weight of about 15356 Daltons was identified in a TB⁺/HIV⁺ sample (gel number P802, protein spot 17). Six peptides (SEQ ID NOs: 48-53 inclusive) matched this protein from the MALDI-TOF data, two with 1 missed cleavage and 4 with no missed cleavages. The percentage coverage of a protein having GenBank Accession No. 053759 (SEQ ID NO: 1) by these 6 peptides (SEQ ID NOs: 48-53) was 32.1%, suggesting that the peptide fragments were derived from the same protein marker. Two peptides had methionine sulfoxide modifications. These data are presented in Table 1 and are extracted from the IonIQ database used to analyse the PMF data.

The identified protein having the amino acid sequence set forth in SEQ ID NO: 1 was designated as “BSX”, based upon the presence of a helix-turn-helix motif generally found in XRE-family like proteins (i.e., transcriptional regulatory proteins that bind to cis-acting xenobiotic response elements in the upstream regions of certain prokaryote genes (e.g., Cro, cI, HipB).

The amino acid sequence set forth in SEQ ID NO: 1 corresponds to a hypothetical protein of M. tuberculosis identified from in silico translation of open reading frames of the M. tuberculosis genome. Accordingly, this provides the first disclosure that the BSX protein of M. tuberculosis is expressed during an active tuberculosis infection in a human host. These data suggest that the M. tuberculosis BSX protein identified in TB⁺/HIV⁺ sera is a good candidate protein for preparation of diagnostic and therapeutic reagents for tuberculosis.

TABLE 1
Identification of a diagnostic marker of M. tuberculosis infection

PeptideID = peptide identification number
DB Mass = theoretical mass of peptide
User Mass = observed mass of peptide
PPM error = error associated with mass of peptide in parts per million
MC = miscleavages
Pep Start = Amino acid number at beginning of peptide
Pep End = Amino acid number at end of peptide
Mods = Modifications
Sequence = Amino acid sequence
Accession number: O53759
Sequence name: PUTATIVE REGULATORY PROTEIN (Transcriptional regulator, PbsX family)
Species: Mycobacterium tuberculosis
Molecular weight: 15356
Isoelectric point: 5.23
MSSEEKLAAK VSTKASDVAS DIGSFIRSQR ETAHVSMRQL AERSGVSNPY LSQVERGLRK PSADVLSQIA
KALRVSAEVL YVRAGILEPS ETSQVRDAII TDTAITERQK QILLDIYASF THQNEATREE CPSDPTPTDD
(SEQ ID NO: 1)
Amino acid coverage: 45 Percentage coverage: 32.14%

| | | | | | | | | |
|---|---|---|---|---|---|---|---|---|
| PeptideID | DB Mass | User Mass | PPM error | MC | Pep Start | Pep End | Mods | Sequence |

| | | | | | | | | |
|---|---|---|---|---|---|---|---|---|
| O53759.06.1.0.10000T | 726.297980 | 726.225000 | 100.4923 | 0 | 1 | 6 | MSO: 1 | MSSEEK |
| | | | | | | | | (SEQ ID NO: 48) |
| O53759.06.31.0.1000T | 946.441620 | 946.506000 | −68.0186 | 0 | 31 | 38 | MSO: 1 | ETAHVSMR |
| | | | | | | | | (SEQ ID NO: 49) |
| O53759.06.15.0.00000T | 1337.670080 | 1337.710000 | −29.8420 | 0 | 15 | 27 | | ASDVASDIGSFIR |
| | | | | | | | | (SEQ ID NO:50) |
| O53759.O6.44.0.00000T | 1435.718110 | 1435.700000 | 12.6141 | 0 | 44 | 56 | | SGVSNPYLSQVER |
| | | | | | | | | (SEQ ID NO: 51) |
| O53759.06.15.1.00000T | 1708.861800 | 1708.850000 | 6.9052 | 1 | 15 | 30 | | ASDVASDIGSFIRSQR |
| | | | | | | | | (SEQ ID NO: 52) |
| O53759.06.39.1.00000T | 2033.041560 | 2032.990000 | 25.3617 | 1 | 39 | 56 | | QLAERSGVSNPYLSQVER |
| | | | | | | | | (SEQ ID NO: 53) |

### Example 4

### B Cell Epitope Mapping of the BSX Protein of M. Tuberculosis

### 1. Synthetic Peptides

To identify immunogenic epitopes of the BSX protein, a set of synthetic peptides (PEPSET) was produced from the primary amino acid sequence shown in SEQ ID NO: 1. The synthetic peptides comprised the amino acid sequences set forth in SEQ ID Nos: 2-44, with additional N-terminal and/or C-terminal sequence extensions added compared to the sequence of the corresponding fragment in SEQ ID NO: 1.

In particular, a synthetic peptide (#1 in Table 2) was produced consisting of the N-terminal 15 residues of SEQ ID NO: 1 with a C-terminal extension Gly-Ser-Gly. The base peptide sequence for this peptide is set forth in SEQ ID NO: 2. The remaining synthetic peptides (i.e., #2 to #43 in Table 2) in the PEPSET contained the N-terminal extension Ser-Gly-Ser-Gly added to the base peptide sequences of BSX set forth in SEQ ID Nos: 3-44, respectively.

The structures of the peptides are set forth in Table 2. In addition to the sequences of the base peptides, i.e., comprising a sequence derived from SEQ ID NO: 1 and an N-terminal and/or C-terminal sequence extension, the structures presented in Table 2 include labels added to the N-terminus and/or C-terminus of each peptide (e.g., biotin, BioCytAm) to facilitate their use in immunoassays e.g., ELISA. In contrast, the sequences set forth in SEQ ID Nos: 2-44 merely show the sequences of the base peptides, i.e., comprising a sequence derived from SEQ ID NO: 1 and an N-terminal and/or C-terminal sequence extension, and do not include the modified residues. The skilled artisan will readily be able to add such modification using standard procedures of peptide synthesis and immunological detection of proteins.

TABLE 2

| | | | | |
|---|---|---|---|---|
| | | | | SEQ ID NO: |
| No. | Peptide structure | Hydra | MolWt | (base peptide) |

| | | | | |
|---|---|---|---|---|
| #1 | Biotin- MSSEEKLAAKVSTKAGSG-BiocytAm | 0.24 | 2,360.82 | 2 |
| #2 | Biotin- SGSGEEKLAAKVSTKASDV-NH2 | 0.07 | 2,089.37 | 3 |
| #3 | Biotin- SGSGLAAKVSTKASDVASD-NH2 | 0.16 | 1,976.21 | 4 |
| #4 | Biotin- SGSGKVSTKASDVASDIGS-NH2 | 0.13 | 1,978.18 | 5 |
| #5 | Biotin- SGSGTKASDVASDIGSFIR-NH2 | 0.25 | 2,080.32 | 6 |
| #6 | Biotin- SGSGSDVASDIGSFIRSQR-NH2 | 0.20 | 2,151.36 | 7 |
| #7 | Biotin- SGSGASDIGSFIRSQRETA-NH2 | 0.18 | 2,151.36 | 8 |
| #8 | Biotin- SGSGIGSFIRSQRETAHVS-NH2 | 0.27 | 2,201.47 | 9 |
| #9 | Biotin- SGSGFIRSQRETAHVSMRQ-NH2 | 0.18 | 2,359.69 | 10 |
| #10 | Biotin- SGSGSQRETAHVSMRQLAE-NH2 | 0.13 | 2,256.52 | 11 |
| #11 | Biotin- SGSGETAHVSMRQLAERSG-NH2 | 0.14 | 2,185.44 | 12 |
| #12 | Biotin- SGSGHVSMRQLAERSGVSN-NH2 | 0.17 | 2,184.46 | 13 |
| #13 | Biotin- SGSGMRQLAERSGVSNPYL-NH2 | 0.27 | 2,234.56 | 14 |
| #14 | Biotin- SGSGLAERSGVSNPYLSQV-NH2 | 0.31 | 2,133.38 | 15 |
| #15 | Biotin- SGSGRSGVSNPYLSQVERG-NH2 | 0.17 | 2,162.39 | 16 |
| #16 | Biotin- SGSGVSNPYLSQVERGLRK-NH2 | 0.20 | 2,259.59 | 17 |
| #17 | Biotin- SGSGPYLSQVERGLRKPSA-NH2 | 0.22 | 2,214.55 | 18 |
| #18 | Biotin- SGSGSQVERGLRKPSADVL-NH2 | 0.17 | 2,168.48 | 19 |
| #19 | Biotin- SGSGERGLRKPSADVLSQI-NH2 | 0.19 | 2,182.50 | 20 |
| #20 | Biotin- SGSGLRKPSADVLSQIAKA-NH2 | 0.25 | 2,110.48 | 21 |
| #21 | Biotin- SGSGPSADVLSQIAKALRV-NH2 | 0.36 | 2,081.44 | 22 |
| #22 | Biotin- SGSGDVLSQIAKALRVSAE-NH2 | 0.29 | 2,113.44 | 23 |
| #23 | Biotin- SGSGSQIAKALRVSAEVLY-NH2 | 0.38 | 2,161.53 | 24 |
| #24 | Biotin- SGSGAKALRVSAEVLYVRA-NH2 | 0.33 | 2,159.56 | 25 |
| #25 | Biotin- SGSGLRVSAEVLYVRAGIL-NH2 | 0.51 | 2,172.60 | 26 |
| #26 | Biotin- SGSGSAEVLYVRAGILEPS-NH2 | 0.42 | 2,117.43 | 27 |
| #27 | Biotin- SGSGVLYVRAGILEPSETS-NH2 | 0.42 | 2,147.45 | 28 |
| #28 | Biotin- SGSGVRAGILEPSETSQVR-NH2 | 0.24 | 2,155.43 | 29 |
| #29 | Biotin- SGSGGILEPSETSQVRDAI-NB2 | 0.27 | 2,128.36 | 30 |
| #30 | Biotin- SGSGEPSETSQVRDAIITD-NH2 | 0.17 | 2,174.34 | 31 |
| #31 | Biotin- SGSGETSQVRDAIITDTAI-NB2 | 0.28 | 2,146.38 | 32 |
| #32 | Biotin- SGSGQVRDAIITDTAITER-NH2 | 0.23 | 2,215.49 | 33 |
| #33 | Biotin- SGSGDAIITDTAITERQKQ-NH2 | 0.17 | 2,216.47 | 34 |
| #34 | Biotin- SGSGITDTAITERQKQILL-NH2 | 0.35 | 2,256.62 | 35 |
| #35 | Biotin- SGSGTAITERQKQILLDIY-NH2 | 0.38 | 2,318.70 | 36 |
| #36 | Biotin- SGSGTERQKQILLDIYASF-NH2 | 0.37 | 2,338.69 | 37 |
| #37 | Biotin- SGSGQKQILLDIYASFTHQ-NH2 | 0.44 | 2,318.66 | 38 |
| #38 | Biotin- SGSGILLDIYASFTHQNEA-NH2 | 0.47 | 2,248.52 | 39 |
| #39 | Biotin- SGSGDIYASFTHQNEATRE-NH2 | 0.15 | 2,295.45 | 40 |
| #40 | Biotin- SGSGASFTHQNEATREECP-NH2 | 0.14 | 2,233.40 | 41 |
| #41 | Biotin- SGSGTHQNEATREECPSDP-NH2 | 0.03 | 2,227.35 | 42 |
| #42 | Biotin- SGSGNEATREECPSDPTPT-NH2 | 0.08 | 2,160.30 | 43 |
| #43 | Biotin- SGSGATREECPSDPTPTDD-OH | 0.06 | 2,148.24 | 44 |

### 2. Serum Samples

A total of 30 TB-positive samples and 52 TB-negative samples were screened with the peptides of the PEPSET. These included sera from South African (S.A.) Zulu TB-positive individuals, S.A. Zulu TB-negative individuals, S.A. Caucasian TB-negative individuals, World Health Organisation (WHO) TB-positive individuals of unknown race, WHO TB-negative individuals of unknown race, and Australian Caucasian TB-negative control individuals and plasma from Chinese TB-positive individuals and Chinese TB-negative individuals.

Samples were screened for the presence of antibodies using an ELISA system developed as described below.

### 3. ELISA Assay

Nunc-Immuno module maxisorp wells were coated overnight at room temperature or at 4° C. over the weekend with 100 μl/well of 5 μg/ml streptavidin diluted in milli-Q water. The streptavidin was flicked out of the wells and each well was blocked with 200 μl phosphate-buffered saline (PBS) containing 1.0% (w/v) casein, 0.1% (v/v) Tween 20 and 0.1% (w/v) Azide (blocker) per well. After 1 hour, the blocker was removed, and each well was coated with 100 μl of biotinylated peptide in blocker for 1 hour, with agitation of the plate. Subsequently, each well was washed 5 times with PBS/0.1% Tween 20, allowed to dry on absorbent paper, and either stored at 4° C. with dessicant, or used immediately. This was followed by incubation for 1 hour with agitation in 50 μl of patient serum or plasma, diluted 1:50 in blocker. Following this incubation, all wells were washed 5 times, using PBS/0.1% Tween 20 in a laminar flow, and tapped dry. Then 100 μl Sheep anti-human IgG Horse Radish Peroxidase (HRP) conjugate was added to each well. The conjugate was diluted 1:10,000 (v/v) in PBS/0.1% (w/v) casein/0.1% (v/v) Tween 20/0.1% (w/v) thimerosal, and incubated for 1 hour with agitation. Each well was then washed 4 times using PBS/0.1% (v/v) Tween 20, and twice using PBS. Finally, 100 μl liquid TMB substrate based system (Sigma) was added to each well, and the wells incubated at room temperature in the dark for 20 mins.

Reactions were stopped by addition of 100 μl 0.5M Sulfuric acid. Each peptide was assayed in duplicate and repeated if duplicates did not appear to be reproducible.

Alongside the patient samples, four control samples were also tested, as follows:
1. Negative control: streptavidin/peptide 24/no serum or plasma/conjugate;
2. Peptide Control: streptavidin/no peptide/patient serum or plasma/conjugate;
3. Positive control: streptavidin/peptide 24/S.A. serum 7/conjugate; and
4. Serum background: no streptavidin/no peptide/patient serum or plasma/conjugate.

S.A. serum 7 was used for the positive control, due to its consistent reproducible positive results found in preliminary ELISA experimentation.

### 4. Data Analysis

Immunogenic peptides represent outliers in the distribution of peptide absorbencies and are detected following log transformation normalisation by calculation of a normal score statistic, with a mean and standard deviation estimated by a robust M-Estimator.

### 5. Results

Mass screening of the TB-positive and TB-negative samples for the presence of antibodies to BSX peptides demonstrated that 47% of TB-positive samples contained anti-BSX antibodies (Table 3). The TB-negative patients that tested positive for any BSX peptide included 1 Chinese, 1 S.A. Caucasian and 1 Australian Caucasian individuals.

TABLE 3
Summary of number of patients with antibodies against BSX protein

| | Number of samples with anti-BSX antibodies | | |
|---|---|---|---|
| TB status of patient | Total population | HIV+ | HIV− |
| TB-positive (TB+) | 14/30 (47%) | 13/17 (76%) | 1/13 (8%) |
| TB-negative (TB−)* | 3/52 (6%) | 0/6 (0%) | 3/46 (7%) |

*none of the control patients that tested positive for BSX also tested positive for BSX peptide 24

Differentiation of the total patient population to include HIV status elucidated a TB/HIV correlation, where 76% of the TB-positive samples that contained anti-BSX antibodies were also HIV⁺ (Table 3). This is further demonstrated in the S.A. group of samples where 80% of the S.A. TB-positive/HIV⁺ samples screened contained antibodies to BSX (Table 4).

TABLE 4
Proportion of South African TB patients (HIV⁺ and HIV⁻)
containing anti-BSX antibodies

| TB status of patient | HIV+ status | HIV− status |
|---|---|---|
| TB-positive (TB+) | 12/15 (80%) | 1/4 (25%) |
| TB-negative (TB−) | 0/5 (0%) | 1/26 (4%)* |

*This includes S.A. Caucasian 1/10 (10%).

Table 5 illustrates the specificity and recurrence of anti-BSX antibodies found in the S.A. TB-positive/HIV⁺ population. A total of 8 BSX peptides were unique to the S.A. TB-positive/HIV⁺ sera samples screened. Anti-BSX 24 antibodies (comprising the base peptide set forth in SEQ ID NO: 25) were found in 9/12 cases, followed by anti-BSX 23 (comprising the base peptide set forth in SEQ ID NO: 24) antibodies occurring in 3/12 cases, anti-BSX 20 (comprising the base peptide set forth in SEQ ID NO: 21) and anti-BSX 35 (comprising the base peptide set forth in SEQ ID NO: 36) found in 2/12 cases and anti-BSX 13 (comprising the base peptide set forth in SEQ ID NO: 14), anti-BSX 19 (comprising the base peptide set forth in SEQ ID NO: 20), anti-BSX 21 (comprising the base peptide set forth in SEQ ID NO: 22) and anti-BSX 42 (comprising the base peptide set forth in SEQ ID NO: 43) found in only 1/12 cases.

TABLE 5
Recurring peptides in S.A. Zulu HIV⁺ sera
containing anti-BSX antibodies

| Immunogenic BSX peptide No. (SEQ ID NO.) | Occurrence |
|---|---|
| BSX 24 (SEQ ID NO: 25) | 9/12 |
| BSX 23 (SEQ ID NO: 24) | 3/12 |
| BSX 20 (SEQ ID NO: 21), BSX 35 (SEQ ID NO: 36) | 2/12 |
| BSX 13 (SEQ ID NO: 14), BSX 19 (SEQ ID NO: 20) | 1/12 |
| BSX 21 (SEQ ID NO: 22), BSX 42 (SEQ ID NO: 43) | |

Conversely, all of the Chinese plasma screened for BSX were HIV⁻ and categorised according to their pulmonary diagnosis (Table 6). None of the extra-pulmonary or pulmonary TB-positive plasma contained antibodies to BSX whilst only one of the TB-negative plasma screened contained anti-BSX antibodies to one BSX peptide.

TABLE 6
Proportion of plasma containing anti-BSX antibodies in Chinese patients
outlining pulmonary status

| TB status of patient | Total | Pulmonary TB | Extra-pulmonary TB |
|---|---|---|---|
| TB-positive (TB+) | 0/9 (0%) | 0/4 (0%) | 0/5 (0%) |
| TB-negative (TB−) | 1/9 (11%) | — | — |

The final TB-positive group screened for anti-BSX antibodies were 4 WHO sera samples of unknown racial origin. Two of the samples were TB-positive/HIV⁺ one of which tested positive for antibodies to BSX, specifically BSX 24 comprising the base peptide set forth in SEQ ID NO: 25.

Antibodies against BSX were identified in 3 TB-negative samples, however none contained antibodies to the eight candidate peptides unique to TB irrespective of racial origin. These include SEQ ID Numbers: 14, 20, 21, 22, 24, 25, 29 and 36.

### 6. Discussion

ELISA analysis of TB positive and TB negative serum or plasma led to the identification of a number of immunogenic BSX peptides containing B cell epitopes of the full-length BSX protein of M. tuberculosis.

Peptide BSX 24 (SEQ ID NO: 25) was not found to be immunogenic in any control TB-negative serum or control plasma tested. Nor were any of the eight unique candidate peptides found to be immunogenic in sera from TB-negative S.A. Zulu subjects, thus reinforcing the suitability of BSX and/or any of its peptides as a diagnostic reagent, and as an immunogen for the preparation of monoclonal antibodies suitable for use in an antigen-based assay for M. tuberculosis infection.

Furthermore, the correlation between HIV status and TB status with respect to serological reactivity of BSX peptide 24 (SEQ ID NO: 25) has many therapeutic advantages, such as, for example, the ability to detect TB and HIV status and/or monitoring the TB status in HIV⁺ individuals. To further emphasise the correlation between TB and HIV, it is important to note that all of the Chinese samples investigated were HIV⁻ negative.

The absence of detectable antibodies against BSX in plasma from patients in a Chinese cohort may be associated with pulmonary TB being confined to the lung, whereas in the South African patients HIV positive status is often associated with extrapulmonary disease, which is more systemic. Alternatively, BSX may not be as highly expressed in Chinese compared to South African TB patients.

### Example 5

### Screening of TB and Non-TB Sera Against Synthetic Peptides Derived from the BSX Protein (SEQ ID NO: 1)

### 1. Synthetic Peptides

Three peptides were synthesised from the amino acid sequence of the Transcriptional regulator, PbsX family (SwissProt entry number O53759) and evaluated as capture agents for Human immunoglobulin G in TB-positive sera. One peptide, designated BSX (23-24) peptide (SEQ ID NO: 45) comprises the sequence of the highly immunogenic peptide set forth in SEQ ID NO: 25 determined in the preceding example, with additional N-terminal and C-terminal sequences flanking this sequence in the full-length protein (SEQ ID NO: 1) and conjugated C-terminally to a cysteine residue. Another peptide, designated N—C terminal (SEQ ID NO: 46) comprised the N-terminal seven residues of SEQ IDS NO: 1 fused to the C-terminal seven residues of SEQ ID NO: 1 by an intervening cysteine residue. A third peptide, designated peptide 28 (SEQ ID NO: 47) comprised the sequence set forth in SEQ ID NO: 29 conjugated C-terminally to a cysteine residue. The sequences of these three additional peptides is presented in Table 7 below:

TABLE 7
Sequences of additional peptides

| | | |
|---|---|---|
| BSX (23-24) | SQIAKALRVSAEVLYVRAC | (SEQ ID NO: 45) |
| peptide | | |
| N-C terminal | MSSEEKLCDPTPTDD | (SEQ ID NO: 46) |
| peptide 28 | VRAGILEPSETSQVRC (SEQ ID NO: 47) | |

For ELISA formats, the peptides set forth in SEQ ID NOs: 45-47 additionally comprised an N-terminal linker (Ser-Gly-Ser-Gly) to the base peptide as described in the preceding example, to facilitate binding of the peptide to solid matrices.

The C-terminal and internal cysteine residues were included to facilitate cross-linking of the peptides for subsequent antibody production.

### 2. Sera

Sera were a panel of sera obtained from 41-44 TB-positive patients (i.e., TB-positive sera) in each experiment, and 51 healthy control subjects (i.e., non-TB sera).

### 3. ELISA Assay

Peptides comprising SEQ ID NOs: 45-47 were coated on ELISA trays at 3 μg/mL on a streptavidin base of 5 μg/mL and then probed (after blocking) with Non-TB control sera and Known TB-positive sera. Sera were diluted 1/50 (v/v) prior to use. Capture of human IgG was traced with enzyme-linked sheep anti-HuIgG/tetramethylbenzidine (TMB) substrate.

### 4. Statistical Analyses

The sensitivity and specificity were analysed by taking the average substrate product OD values (from the conjugated peroxidase/TMB reaction) and calculating the cut-off values for significance at two standard deviations above the average and three standard deviations above the mean (i.e., at the 95% and 99.7% significance levels, respectively). For the control sera, one sample produced an outlier OD value by Dixon's outlier test (N=30). The analyses were compared including or excluding this outlier.

As used herein, term “sensitivity” in the context of a diagnostic/prognostic assay is understood to mean the proportion of TB-positive subjects that are diagnosed using a particular assay method (i.e., a “true” positive). Accordingly, an assay that has increased sensitivity is capable of detecting a greater proportion of TB-infected subjects than an assay with reduced or lower sensitivity.

As used herein, the term “specificity” in the context of a diagnostic/prognostic assay is understood to mean the proportion of non-TB subjects (i.e., non-infected subjects) that do not return a positive result using a particular assay method (i.e., “true” negatives). Accordingly, an assay that has increased or enhanced specificity returns fewer false positive results or is capable of distinguishing between infected and non-infected subjects to a greater degree than an assay with a reduced specificity.

### 4. Results

### a) BSX (23-24) Peptide (SEQ ID NO: 45)

BSX (23-24) peptide sequence showed a significant binding to confirmed TB-positive sera. Summary sensitivity and specificity data are presented in Table 8. Data provided for each subject in Table 8 indicate that a peptide comprising the sequence set forth in SEQ ID NO: 45 selectively identifies antibodies against M. tuberculosis in patient sera. Data also show that the sensitivity and specificity with these revised criteria are relatively unchanged irrespective of whether or not the outliers is omitted, however there is a marginal increase in sensitivity at the 3 standard deviation level.

TABLE 8
Sensitivity and specificity data for BSX (23-24) peptide (SEQ ID NO: 45)

| | % assay value | | % assay value | |
|---|---|---|---|---|
| | including control | | omitting control | |
| | sera outlier | | sera outlier | |
| | 2 σ | 3 σ | 2 σ | 3 σ |
| Assay Sensitivity for | 46.3% | 43.9% | 48.8% | 46.3% |
| detecting TB-positive | | | | |
| sera (n = 41) | | | | |
| Assay Specificity for not | 94.1% | 98.0% | 92.0% | 98.0% |
| detecting TB-negative | | | | |
| sera (n = 51) | | | | |

b) N—C terminal (SEQ ID NO: 46) and Peptide 28 (SEQ ID NO: 47)

These two peptides showed only weak interaction against a range of confirmed TB-positive sera. As shown in Tables 9 and 10, assays using these peptides were not highly sensitive, albeit specific in so far as they omit false positive detection.

TABLE 9
Sensitivity and specificity data for N—C Terminal peptide
(SEQ ID NO: 46)

| | % assay value | | % assay value | |
|---|---|---|---|---|
| | including control | | omitting | |
| | sera outlier | | control sera outlier | |
| | 2 σ | 3 σ | 2 σ | 3 σ |
| Assay Sensitivity for | 13.3% | 6.7% | 17.8% | 8.9% |
| detecting TB-positive | | | | |
| sera (n = 41) | | | | |
| Assay Specificity for not | 95.8% | 95.8% | 93.6% | 95.7% |
| detecting TB-negative | | | | |
| sera (n = 51) | | | | |

TABLE 10
Sensitivity and specificity data for peptide 28 (SEQ ID NO: 47)

| | % assay value | | % assay value | |
|---|---|---|---|---|
| | including control | | omitting | |
| | sera outlier | | control sera outlier | |
| | 2 σ | 3 σ | 2 σ | 3 σ |
| Assay Sensitivity for | 11.1% | 4.4% | Not done | Not done |
| detecting TB-positive | | | | |
| sera (n = 41) | | | | |
| Assay Specificity for not | 94.1% | 96.1% | Not done | Not done |
| detecting TB-negative | | | | |
| sera (n = 51) | | | | |

The data presented in Tables 8-10 indicate that BSX (23-24) peptide (SEQ ID NO: 45) has utility in antibody-based assays to detected tuberculosis in patient samples, especially sera. The other two peptides tested in this example (SEQ ID NOs: 46 and/or 47) also have utility in eliminating false positive detection e.g., as part of a multi-analyte test alongside SEQ ID NO: 45. This finding is not particularly surprising in view of the data presented in the preceding example, demonstrating the peptides 23 and 24 (i.e., SEQ ID Nos: 24 and 25, respectively) and particularly SEQ ID NO: 25 included in the sequence of SEQ ID NO: 45 consist of or comprise immunogenic B cell epitopes of the full length BSX protein.

### Example 6

### Screening of TB and Non-TB Sera Against Recombinant Full-Length BSX protein (SEQ ID NO: 1)

### 1. Sera

The sera panels were from 44 TB-positive (smear or culture) Chinese and South African patients (i.e., TB-positive sera), and 44 healthy control subjects (i.e., non-TB sera).

### 2. ELISA Assay

Recombinant BSX protein (SEQ ID NO: 1) was coated directly onto ELISA trays at 5 μg/mL and then probed (after blocking) with Non-TB control sera and known TB-positive sera diluted 1/100 (v/v) in buffer. Capture of human IgG was traced with enzyme-linked sheep anti-HuIgG/tetramethylbenzidine (TMB) substrate.

### 3. Statistical Analyses

The sensitivity and specificity were analysed by taking the average substrate product OD values (from the conjugated peroxidase/TMB reaction) and calculating the cut-off values for significance at two standard deviations above the average and three standard deviations above the mean (i.e., at the 95% and 99.7% significance levels, respectively).

### 4. Results

As shown in Table 11, recombinant BSX protein assayed under these conditions was highly specific in detecting TB-positive sera. Sensitivity of the assay over the populations tested was intermediate between SEQ ID NO: 45 and SEQ ID NOs: 46-47.

On the other hand, the sensitivity of the assay in South African TB sera smears or culture positives is higher than the overall sensitivity (i.e., 35% compared to 25% at three standard deviations cut-off value). Using Chinese smear or culture TB sera, the sensitivity of the assay is lower than the overall sensitivity (i.e., 11% compared to 25% at three standard deviations cut-off value). In both Chinese and South African populations, the specificity of the assay is 100%, indicating robustness in this parameter.

TABLE 11
Sensitivity and specificity data for recombinant BSX protein
(SEQ ID NO: 1)

| | % assay value | | % assay value | |
|---|---|---|---|---|
| | including control | | omitting control | |
| | sera outlier | | sera outlier | |
| | 2 σ | 3 σ | 2 σ | 3 σ |
| Assay Sensitivity for | 29.5% | 25.0% | Not done | Not done |
| detecting TB-positive | | | | |
| sera (n = 44) | | | | |
| Assay Specificity for not | 95.5% | 100.0% | Not done | Not done |
| detecting TB-negative | | | | |
| sera (n = 44) | | | | |

### Example 7

### Screening of TB and Non-TB Sera According to HIV Status

### 1. Sera

Sera were a panel of sera obtained from the following subjects:

(i) Five (5) TB-positive and HIV-negative smear or culture South African patients (i.e., TB⁺HIV⁻ sera);
(ii) Twenty one (21) TB-positive and HIV-positive smear or culture South African patients (i.e., TB⁺HIV⁺ sera); and
(iii) Twenty (20) TB-negative and HIV-negative smear or culture subjects (i.e., healthy control sera).

### 2. ELISA Assay

Recombinant BSX protein (SEQ ID NO: 1) or BSX (23-24) peptide (SEQ ID NO: 45) was coated directly onto ELISA trays at 5 μg/mL and then probed (after blocking) with Non-TB control sera and known TB-positive sera diluted 1/100 (v/v) in buffer. Alternatively, the BSX(23-24) peptide was used as described in the preceding examples. Capture of human IgG was traced with enzyme-linked sheep anti-HuIgG/tetramethylbenzidine (TMB) substrate.

### 3. Statistical Analyses

The sensitivity and specificity were analysed by taking the average substrate product OD values (from the conjugated peroxidase/TMB reaction) and calculating the cut-off values for significance at two standard deviations above the average and three standard deviations above the mean (i.e., at the 95% and 99.7% significance levels, respectively).

### 4. Results

As shown in Table 12, recombinant BSX protein assayed under these conditions was highly specific in detecting TB-positive sera. Sensitivity of the assay over the populations tested was also quite high for HIV⁺ patients. Similar results were obtained using the BSX(23-24) peptide. Thus, the full-length recombinant BSX protein and BSX(23-24) peptide separately detect about 40-45% of TB⁺ HIV⁺ subjects, and, in a multianalyte test format, detect about 65% to 70% of TB⁺HIV⁺ subjects, with only about 5% false-positive detection.

On the other hand, the sensitivity of the assay in South African TB sera smears or culture positives is higher than the overall sensitivity (i.e., 35% compared to 25% at three standard deviations cut-off value). Using Chinese smear or culture TB sera, the sensitivity of the assay is lower than the overall sensitivity (i.e., 11% compared to 25% at three standard deviations cut-off value). In both Chinese and South African populations, the specificity of the assay is absolute i.e., 100% indicating robustness in this parameter.

TABLE 12
Sensitivity and specificity data for recombinant BSX protein
(SEQ ID NO: 1)

| | Recombinant | BSX (23-24) | Combined |
|---|---|---|---|
| | BSX (SEQ | peptide | (protein and |
| | ID NO: 1) | (SEQ ID NO: 45) | peptide) |
| Assay Sensitivity for | 0% | 0% | 0% |
| detecting TB⁺HIV⁻ sera | | | |
| (n = 5) | | | |
| Assay Sensitivity for | 40% | 43% | 67% |
| not detecting TB⁺HIV⁺ | | | |
| sera (n = 21) | | | |
| Assay Sensitivity for | 31% | 35% | 54% |
| combined TB⁺HIV⁺ | | | |
| and TB⁺HIV⁻ sera | | | |
| (n = 26) | | | |
| Assay Sensitivity for | 0% | 5% | 5% |
| healthy control sera | | | |
| (n = 20) | | | |
| Assay Specificity for | 100% | 95% | 95% |
| not detecting TB⁺ sera | | | |
| (n = 20) | | | |

These data indicate that the full-length BSX protein, e.g., expressed as a recombinant protein, can be used in combination with a synthetic peptide comprising the dominant B-cell epitope identified herein e.g., peptide 24 (SEQ ID NO: 25) or BSX(23-24) peptide (SEQ ID NO: 45), to diagnose the presence of an active infection or recent past infection by M. tuberculosis.

For example, recombinant full-length BSX (SEQ ID NO: 1) and BSX(23-24) peptide are both biotinylated and immobilized onto a streptavidin base (5 μg/ml) that has been preadsorbed onto wells of a microtitre pl ate. Standard ELISA reactions are carried out wherein (i) patient sera and control sera, each diluted 1/100 (v/v) in buffer, are added to separate wells, and (ii) capture of human IgG in the sera by the immobilized protein and peptide is traced using enzyme-linked sheep anti-HuIgG detected using tetramethylbenzidine (TMB) substrate.

1. An isolated or recombinant immunogenic BSX protein of Mycobacterium tuberculosis or an immunogenic BSX peptide or immunogenic BSX fragment or epitope thereof.

2. The isolated or recombinant immunogenic BSX protein of M. tuberculosis according to claim 1 comprising the amino acid sequence set forth in SEQ ID NO: 1 or having an amino acid sequence that is at least about 95% identical to SEQ ID NO: 1.

3.-13. (canceled)

14. The immunogenic BSX peptide or immunogenic BSX fragment or epitope according to claim 1 comprising an amino acid sequence selected from the group consisting of SEQ ID NOs: 14, 20, 21, 22, 24, 25, 36, 45, 46 and 47.

15. The isolated or recombinant immunogenic BSX protein of Mycobacterium tuberculosis or an immunogenic BSX peptide or immunogenic BSX fragment or epitope thereof according to claim 1 wherein said protein, peptide, fragment or epitope comprises one or more labels or detectable moieties to facilitate detection or isolation or immobilization.

16. (canceled)

17. A fusion protein comprising one or more immunogenic BSX peptides, fragments or epitopes according to claim 1.

18.-23. (canceled)

24. An isolated or recombinant antibody or immune reactive fragment of an antibody that binds specifically to the isolated or recombinant immunogenic BSX protein of Mycobacterium tuberculosis or an immunogenic BSX peptide or immunogenic BSX fragment or epitope thereof according to claim 1.

25. The isolated antibody of claim 24 wherein the antibody is a polyclonal antibody.

26. The isolated antibody of claim 24 wherein the antibody is a monoclonal antibody.

27. An isolated antibody-producing cell or antibody-producing cell population that produces the monoclonal antibody of claim 26.

28.-31. (canceled)

32. A method of diagnosing tuberculosis or an infection by M. tuberculosis in a subject comprising detecting in a biological sample from said subject antibodies against an immunogenic BSX protein or an immunogenic BSX peptide or immunogenic BSX fragment or epitope thereof, wherein the presence of said antibodies in the sample is indicative of infection.

33. The method of claim 32 comprising contacting a biological sample derived from the subject with the isolated or recombinant immunogenic BSX protein of Mycobacterium tuberculosis or an immunogenic BSX peptide or immunogenic BSX fragment or epitope thereof according to claim 1 for a time and under conditions sufficient for an antigen-antibody complex to form and then detecting the formation of an antigen-antibody complex.

34. The method of claim 33 wherein detecting the formation of an antigen-antibody complex comprises detecting human immunoglobulin in the antigen-antibody complex.

35. The method of claim 34 wherein detecting human immunoglobulin comprises contacting the antigen-antibody complex with a second antibody comprising anti-human immunoglobulin for a time and under conditions sufficient for said second antibody to bind to the human immunoglobulin in the complex and then detecting the bound anti-human immunoglobulin.

36. The method of claim 35 wherein the second antibody is labelled with a detectable marker or reporter molecule.

37. The method of claim 33 wherein the biological sample derived from the subject is contacted with the isolated or recombinant immunogenic BSX protein of Mycobacterium tuberculosis, said protein comprising an amino acid sequence set forth in SEQ ID NO: 1.

38. The method of claim 33 wherein the biological sample derived from the subject is contacted with an immunogenic BSX peptide comprising an amino acid sequence selected from the group consisting of SEQ ID NOs: 14, 20, 21, 22, 24, 25, 36, 45, 46 and 47.

39.-42. (canceled)

43. The method of claim 32 wherein in the subject is Chinese or South African.

44. The method of claim 32 wherein the subject is immune compromised or immune deficient.

45. The method of claim 32 wherein the subject is HTV⁺.

46. The method of claim 32 wherein the sample comprises blood or serum or an immunoglobulin fraction obtained from the subject.

47. A method of diagnosing tuberculosis or infection by M. tuberculosis in a subject comprising detecting in a biological sample from said subject an immunogenic BSX protein or an immunogenic BSX peptide or immunogenic BSX fragment or epitope thereof, wherein the presence of said protein or immunogenic fragment or epitope in the sample is indicative of disease, disease progression or infection.

48. The method of claim 47 wherein said method comprises contacting a biological sample derived from the subject with one or more antibodies capable of binding to a BSX protein or an immunogenic fragment or epitope thereof, and detecting the formation of an antigen-antibody complex.

49. The method of claim 48 wherein an antibody is an isolated or recombinant antibody or immune reactive fragment of an antibody that binds specifically to the isolated or recombinant immunogenic BSX protein of Mycobacterium tuberculosis or an immunogenic BSX peptide or immunogenic BSX fragment or epitope thereof according to claim 2.

50. The method of claim 48 or 49 wherein an antibody is a polyclonal antibody.

51. The method of claim 48 or 49 wherein an antibody is a monoclonal antibody.

52. The method of claim 47 wherein the subject is immune compromised or immune deficient.

53. The method of claim 47 wherein the subject is HIV+.

54. The method of claim 47 wherein the sample comprises an extract from a tissue selected from the group consisting of brain, breast, ovary, lung, colon, pancreas, testes, liver, muscle, bone and mixtures thereof.

55. The method of claim 47 wherein the sample comprises body fluid selected from the group consisting of sputum, serum, plasma, whole blood, saliva, urine, pleural fluid and mixtures thereof.

56. The method of claim 47 wherein the sample is derived from body fluid selected from the group consisting of sputum, serum, plasma, whole blood, saliva, urine, pleural fluid and mixtures thereof.

57. The method of claim 47 comprising contacting a biological sample derived from the subject with an antibody that binds to an immunogenic BSX peptide comprising an amino acid sequence set forth in SEQ ID NO: 45 and with an antibody that binds to an immunogenic BSX protein of Mycobacterium tuberculosis comprising an amino acid sequence set forth in SEQ ID NO: 1 for a time and under conditions sufficient for antigen-antibody complexes to form and then detecting the formation of the antigen-antibody complexes.

58. The method of claim 48 further comprising contacting a biological sample derived from the subject with an antibody that binds to an immunogenic protein or peptide of Mycobacterium tuberculosis other than isolated or recombinant immunogenic BSX protein or an immunogenic BSX peptide or immunogenic BSX fragment or epitope thereof.

59.-60. (canceled)

61. A method for determining the response of a subject having tuberculosis or an infection by M. tuberculosis to treatment with a therapeutic compound for said tuberculosis or infection, said method comprising detecting a BSX protein or an immunogenic fragment or epitope thereof in a biological sample from said subject, wherein a level of the protein or fragment or epitope that is enhanced compared to the level of that protein or fragment or epitope detectable in a normal or healthy subject indicates that the subject is not responding to said treatment or has not been rendered free of disease or infection and wherein a level of the protein or fragment or epitope that is lower than the level of the protein or fragment or epitope detectable in a subject suffering from tuberculosis or infection by M. tuberculosis indicates that the subject is responding to said treatment or has been rendered free of disease or infection.

62.-88. (canceled)

89. A method of monitoring disease progression, responsiveness to therapy or infection status by M. tuberculosis in a subject comprising determining the level of a BSX protein or an immunogenic fragment or epitope thereof in a biological sample from said subject at different times, wherein a change in the level of the BSX protein, fragment or epitope indicates a change in disease progression, responsiveness to therapy or infection status of the subject.

90.-107. (canceled)

108. A method of treatment of tuberculosis or infection by M. tuberculosis comprising:
(i) performing the method according to claim 32 or 47 thereby detect the presence of M. tuberculosis infection in a biological sample from a subject; and
(ii) administering a therapeutically effective amount of a pharmaceutical composition to reduce the number of pathogenic bacilli in the lung, blood or lymph system of the subject.

109.-114. (canceled)

115. A kit for detecting M. tuberculosis infection in a biological sample, said kit comprising:
(i) one or more isolated antibodies or immune reactive fragments thereof that bind specifically to the isolated or recombinant immunogenic BSX protein of Mycobacterium tuberculosis or an immunogenic BSX peptide or immunogenic BSX fragment or epitope thereof according to claim 1 or 2; and
(ii) means for detecting the formation of an antigen-antibody complex, optionally packaged with instructions for use.

116. A kit for detecting M. tuberculosis infection in a biological sample, said kit comprising:
(i) isolated or recombinant immunogenic BSX protein of Mycobacterium tuberculosis or an immunogenic BSX peptide or immunogenic BSX fragment or epitope thereof according to claim 1 or 2; and
(ii) means for detecting the formation of an antigen-antibody complex, optionally packaged with instructions for use.

117. A solid matrix having adsorbed thereto an isolated or recombinant BSX protein or an immunogenic BSX peptide or immunogenic BSX fragment or epitope thereof according to claim 1 or 2 or a fusion protein comprising said immunogenic BSX protein, peptide, fragment or epitope.

118.-123. (canceled)

124. A solid matrix having adsorbed thereto an antibody that binds to an isolated or recombinant BSX protein or an immunogenic BSX peptide or immunogenic BSX fragment or epitope thereof according to claim 1 or 2.

125.-130. (canceled)


SEQUENCE LISTING (55 sequences)

SEQ ID NO: 1 - protein, 140 aa, M. tuberculosis Bsx protein
MSSEEKLAAKVSTKASDVASDIGSFIRSQRETAHVSMRQLAERSGVSNPYLSQVERGLRKPSADVLSQIAKALRVSAEVLYVRAGILEPSETSQVRDAIITDTAITERQKQILLDIYASFTHQNEATREECPSDPTPTDD

SEQ ID NO: 2 - protein, 15 aa, artificial sequence
  synthetic peptide derived from SEQ ID NO 1 sequence
MSSEEKLAAKVSTKA

SEQ ID NO: 3 - protein, 15 aa, artificial sequence
  synthetic peptide derived from SEQ ID NO 1 sequence
EEKLAAKVSTKASDV

SEQ ID NO: 4 - protein, 15 aa, artificial sequence
  synthetic peptide derived from SEQ ID NO 1 sequence
LAAKVSTKASDVASD

SEQ ID NO: 5 - protein, 15 aa, artificial sequence
  synthetic peptide derived from SEQ ID NO 1 sequence
KVSTKASDVASDIGS

SEQ ID NO: 6 - protein, 15 aa, artificial sequence
  synthetic peptide derived from SEQ ID NO 1 sequence
TKASDVASDIGSFIR

SEQ ID NO: 7 - protein, 15 aa, artificial sequence
  synthetic peptide derived from SEQ ID NO 1 sequence
SDVASDIGSFIRSQR

SEQ ID NO: 8 - protein, 15 aa, artificial sequence
  synthetic peptide derived from SEQ ID NO 1 sequence
ASDIGSFIRSQRETA

SEQ ID NO: 9 - protein, 15 aa, artificial sequence
  synthetic peptide derived from SEQ ID NO 1 sequence
IGSFIRSQRETAHVS

SEQ ID NO: 10 - protein, 15 aa, artificial sequence
  synthetic peptide derived from SEQ ID NO 1 sequence
FIRSQRETAHVSMRQ

SEQ ID NO: 11 - protein, 15 aa, artificial sequence
  synthetic peptide derived from SEQ ID NO 1 sequence
SQRETAHVSMRQLAE

SEQ ID NO: 12 - protein, 15 aa, artificial sequence
  synthetic peptide derived from SEQ ID NO 1 sequence
ETAHVSMRQLAERSG

SEQ ID NO: 13 - protein, 15 aa, artificial sequence
  synthetic peptide derived from SEQ ID NO 1 sequence
HVSMRQLAERSGVSN

SEQ ID NO: 14 - protein, 15 aa, artificial sequence
  synthetic peptide derived from SEQ ID NO 1 sequence
MRQLAERSGVSNPYL

SEQ ID NO: 15 - protein, 15 aa, artificial sequence
  synthetic peptide derived from SEQ ID NO 1 sequence
LAERSGVSNPYLSQV

SEQ ID NO: 16 - protein, 15 aa, artificial sequence
  synthetic peptide derived from SEQ ID NO 1 sequence
RSGVSNPYLSQVERG

SEQ ID NO: 17 - protein, 15 aa, artificial sequence
  synthetic peptide derived from SEQ ID NO 1 sequence
VSNPYLSQVERGLRK

SEQ ID NO: 18 - protein, 15 aa, artificial sequence
  synthetic peptide derived from SEQ ID NO 1 sequence
PYLSQVERGLRKPSA

SEQ ID NO: 19 - protein, 15 aa, artificial sequence
  synthetic peptide derived from SEQ ID NO 1 sequence
SQVERGLRKPSADVL

SEQ ID NO: 20 - protein, 15 aa, artificial sequence
  synthetic peptide derived from SEQ ID NO 1 sequence
ERGLRKPSADVLSQI

SEQ ID NO: 21 - protein, 15 aa, artificial sequence
  synthetic peptide derived from SEQ ID NO 1 sequence
LRKPSADVLSQIAKA

SEQ ID NO: 22 - protein, 15 aa, artificial sequence
  synthetic peptide derived from SEQ ID NO 1 sequence
PSADVLSQIAKALRV

SEQ ID NO: 23 - protein, 15 aa, artificial sequence
  synthetic peptide derived from SEQ ID NO 1 sequence
DVLSQIAKALRVSAE

SEQ ID NO: 24 - protein, 15 aa, artificial sequence
  synthetic peptide derived from SEQ ID NO 1 sequence
SQIAKALRVSAEVLY

SEQ ID NO: 25 - protein, 15 aa, artificial sequence
  synthetic peptide derived from SEQ ID NO 1 sequence
AKALRVSAEVLYVRA

SEQ ID NO: 26 - protein, 15 aa, artificial sequence
  synthetic peptide derived from SEQ ID NO 1 sequence
LRVSAEVLYVRAGIL

SEQ ID NO: 27 - protein, 15 aa, artificial sequence
  synthetic peptide derived from SEQ ID NO 1 sequence
SAEVLYVRAGILEPS

SEQ ID NO: 28 - protein, 15 aa, artificial sequence
  synthetic peptide derived from SEQ ID NO 1 sequence
VLYVRAGILEPSETS

SEQ ID NO: 29 - protein, 15 aa, artificial sequence
  synthetic peptide derived from SEQ ID NO 1 sequence
VRAGILEPSETSQVR

SEQ ID NO: 30 - protein, 15 aa, artificial sequence
  synthetic peptide derived from SEQ ID NO 1 sequence
GILEPSETSQVRDAI

SEQ ID NO: 31 - protein, 15 aa, artificial sequence
  synthetic peptide derived from SEQ ID NO 1 sequence
EPSETSQVRDAIITD

SEQ ID NO: 32 - protein, 15 aa, artificial sequence
  synthetic peptide derived from SEQ ID NO 1 sequence
ETSQVRDAIITDTAI

SEQ ID NO: 33 - protein, 15 aa, artificial sequence
  synthetic peptide derived from SEQ ID NO 1 sequence
QVRDAIITDTAITER

SEQ ID NO: 34 - protein, 15 aa, artificial sequence
  synthetic peptide derived from SEQ ID NO 1 sequence
DAIITDTAITERQKQ

SEQ ID NO: 35 - protein, 15 aa, artificial sequence
  synthetic peptide derived from SEQ ID NO 1 sequence
ITDTAITERQKQILL

SEQ ID NO: 36 - protein, 15 aa, artificial sequence
  synthetic peptide derived from SEQ ID NO 1 sequence
TAITERQKQILLDIY

SEQ ID NO: 37 - protein, 15 aa, artificial sequence
  synthetic peptide derived from SEQ ID NO 1 sequence
TERQKQILLDIYASF

SEQ ID NO: 38 - protein, 15 aa, artificial sequence
  synthetic peptide derived from SEQ ID NO 1 sequence
QKQILLDIYASFTHQ

SEQ ID NO: 39 - protein, 15 aa, artificial sequence
  synthetic peptide derived from SEQ ID NO 1 sequence
ILLDIYASFTHQNEA

SEQ ID NO: 40 - protein, 15 aa, artificial sequence
  synthetic peptide derived from SEQ ID NO 1 sequence
DIYASFTHQNEATRE

SEQ ID NO: 41 - protein, 15 aa, artificial sequence
  synthetic peptide derived from SEQ ID NO 1 sequence
ASFTHQNEATREECP

SEQ ID NO: 42 - protein, 15 aa, artificial sequence
  synthetic peptide derived from SEQ ID NO 1 sequence
THQNEATREECPSDP

SEQ ID NO: 43 - protein, 15 aa, artificial sequence
  synthetic peptide derived from SEQ ID NO 1 sequence
NEATREECPSDPTPT

SEQ ID NO: 44 - protein, 15 aa, artificial sequence
  synthetic peptide derived from SEQ ID NO 1 sequence
ATREECPSDPTPTDD

SEQ ID NO: 45 - protein, 19 aa, artificial sequence
  synthetic peptide derived from SEQ ID NO 1 sequence coupled to cysteine
SQIAKALRVSAEVLYVRAC

SEQ ID NO: 46 - protein, 15 aa, artificial sequence
  synthetic fusion peptide comprising N-terminal and C-terminal portions of SEQ ID NO 1 coupled by internal cysteine residue
MSSEEKLCDPTPTDD

SEQ ID NO: 47 - protein, 16 aa, artificial sequence
  synthetic peptide derived from SEQ ID NO 1 sequence coupled to cysteine residue
VRAGILEPSETSQVRC

SEQ ID NO: 48 - protein, 6 aa, artificial sequence
  sequence of MALDI-TOF peptide fragment present in TB+/HIV+ sample also present in SEQ ID NO 1
MSSEEK

SEQ ID NO: 49 - protein, 8 aa, artificial sequence
  sequence of MALDI-TOF peptide fragment present in TB+/HIV+ sample also present in SEQ ID NO 1
ETAHVSMR

SEQ ID NO: 50 - protein, 13 aa, artificial sequence
  sequence of MALDI-TOF peptide fragment present in TB+/HIV+ sample also present in SEQ ID NO 1
ASDVASDIGSFIR

SEQ ID NO: 51 - protein, 13 aa, artificial sequence
  sequence of MALDI-TOF peptide fragment present in TB+/HIV+ sample also present in SEQ ID NO 1
SGVSNPYLSQVER

SEQ ID NO: 52 - protein, 16 aa, artificial sequence
  sequence of MALDI-TOF peptide fragment present in TB+/HIV+ sample also present in SEQ ID NO 1
ASDVASDIGSFIRSQR

SEQ ID NO: 53 - protein, 18 aa, artificial sequence
  sequence of MALDI-TOF peptide fragment present in TB+/HIV+ sample also present in SEQ ID NO 1
QLAERSGVSNPYLSQVER

SEQ ID NO: 54 - protein, 26 aa, artificial sequence
  immuogenic peptide derived from M. tuberculosis glutamine synthetase
RGTDGSAVFADSNGPHGMSSMFRSFC

SEQ ID NO: 55 - protein, 21 aa, artificial sequence
  immuogenic peptide derived from M. tuberculosis glutamine synthetase
WASGYRGLTPASDYNIDYAIC